(19) United States
(12) Patent Application Publication
(10) Pub. No.: US 2009/0023162 A1
(43) Pub. Date: Jan. 22, 2009

(54) METHOD FOR THE IDENTIFICATION OF ATYPICAL P-ANCA

(75) Inventors: Birgit Terjung, Swisttal (DE); Ulrich Spengler, Bonn (DE)

Correspondence Address: JOYCE VON NATZMER;PEQUIGNOT + MYERS LLC, 200 Madison Avenue, Suite 1901, New York, NY, 10016, US

(73) Assignee: RHEINISCHE FRIEDRICH-WILHELMS UNIVERSITAET BONN, Bonn (DE)

(21) Appl. No.: 11/573,894

(22) PCT Filed: Aug. 19, 2005
(86) PCT No.: PCT/EP2005/054104

§ 371 Date: Oct. 1, 2008

(30) Foreign Application Priority Data
Aug. 20, 2004 (DE) 102004040370.8

(51) Int. Cl. G01N 33/566; G01N 33/536; C07K 2/00; C07H 21/04; G01N 33/573; C12N 15/63; C12N 5/06; C12P 21/02

(52) U.S. Cl. 435/7.32; 436/536; 530/300; 536/23.1; 435/320.1; 435/325; 435/69.1; 435/7.9; 435/7.92

(57) ABSTRACT

The invention relates to a method for the identification of atypical p-ANCA, kits suitable for the same and application of said method to the diagnosis of chronic inflammatory intestinal diseases and autoimmune liver diseases.

The invention relates to methods for the identification of atypical a-ANCA, kits suitable for the same and application of said method to the diagnosis of chronic inflammatory intestinal diseases and autoimmune liver diseases.

### BACKGROUND OF THE INVENTION

Antigen-antibody reactions present a frequently discussed pathogenetic principle during the development of autoimmune diseases. A widely noticed family of autoantibodies are the so-called anti-neutrophil cytoplasmic antibodies (ANCA) (Wiik, A., APMIS 97(suppl.6):12-13 (1989); Savige, J. et al., Am. J. Clin. Pathol. 111:507-513 (1999)). In the last several years, it could be observed that these autoantibodies do not only play an important role in systemic vasculitides like, for example, Wegener's granulomatosis and microscopic polyangiitis, where they are already established seromarkers (Woude van der, F. J. et al., Lancet 1:425-429 (1985), Falk, R. J. und Jennette, J. C., N. Engl. J. Med., 318:1651-1657 (1988)), but that they can also be detected with a high prevalence (70-96%) in the serum of patients with autoimmune liver diseases like, for example, autoimmune hepatitis (AIH) or primary sclerosing cholangitis (PSC) or chronic inflammatory intestinal diseases, for example ulcerative colitis (UC) (Falk, R. J. und Jennette, J. C., New Engl. J. Med. 318:1651-1657 (1988); Duerr, R. H. et al., Gastroenterology 100:1385-1391 (1991); Hardarson, S. et al., Am. J. Clin. Pathol. 99:277-281 (1993); Mulder, A. H. L. et al., Hepatology 17:411-417 (1993); Mulder, A. H. L et al., Clin. Exp. Immunol. 95:490-497 (1994); Targan, S. et al., Gastroenterology 108:1159-1166 (1995); Bansi, D. et al., J. Hepatol. 24:581-586 (1996); Zauli, D. et al., Hepatology 25:1105-1107 (1997); Roozendaal, C. et al., J. Hepatol. 32:734-741 (2000)). In contrast to the systemic vasculitides, the antigen(s) of the ANCA for the autoimmune liver diseases and the chronic inflammatory intestinal diseases have not yet been identified despite intensive efforts all over the world.

The indirect immunofluorescence microscopy on the basis of ethanol-fixed neutrophil granulocytes is the standard detection method for ANCA (Wiik, A., APMIS 97(suppl.6): 12-13 (1989)). There are three different staining patterns: c-ANCA with a diffuse cytoplasmic fluorescence as well as two different “perinuclear” staining patterns (p-ANCA) (Savige, J. et al., Am. J. Clin. Pathol. 111:507-513 (1999)). With the help of the double immunofluorescence staining procedure, it could be proved that the original definition of ANCA as anti-neutrophil cytoplasmic antibodies cannot be applied to p-ANCA at autoimmune liver diseases or UC (Billing, P. et al., Am. J. Pathol. 147:979-987 (1995); Terjung, B. et al., Hepatology 28:332-340 (1998); Mallolas, J. et al., Gut 47:74-78 (2000)). In contrast to the “classic” p-ANCA, which is characterized by a homogeneous, annular staining pattern in the perinuclear cytoplasm, the “atypical” p-ANCA, which is almost exclusively found in patients with autoimmune liver diseases or chronic inflammatory intestinal diseases, shows an inhomogeneous annular staining pattern of the nuclear periphery connected with a characteristic intranuclear mottled pattern (Terjung, B. et al., Hepatology 28:332-340 (1998); Terjung, B. et al., Clin. Exp. Immunol. 126:37-46 (2001)). Analyses under the electron microscope indicated that this intranuclear mottled pattern most likely corresponds to sections of the stained nuclear periphery in the area of nuclear inversions of the multiple-segment nuclear neutrophil granulocytes; thus, a nuclear antigen of the “atypical” p-ANCA can be assumed. To separate these p-ANCA from the “classic” p-ANCA, they have been denominated as “atypical” p-ANCA or p-ANNA (anti-neutrophil nuclear antibodies) (Terjung, B. et al., Gastroenterology 119:310-322 (2000); Terjung, B. et al., Clin. Exp. Immunol. 126:37-46 (2001)).

In direct accordance with this observation is the fact that “atypical” p-ANCA reacted in not more than 25-35% of the sera with the so far suggested neutrophil-specific granular or cytosolic proteins (like, for example, bactericidal/permeability increasing protein, cathepsin G, elastase, lactoferrin, catalase, enolase) (Zhao, M. H. et al., Clin. Exp. Immunol. 99:49-56 (1995); Halbwachs-Mecarelli, L. et al., Clin. Exp. Immunol. 90:79-84 (1992); Peen, E. et al., Gut 34:56-62 (1993); Stoffel, M. P. et al., Clin. Exp. Immunol. 104:54-59 (1996); Walmsley, R. S: et al., Gut 40:105-109 (1997); Orth, T. et al., Clin. Exp. Immunol. 112:507-515 (1998); Roozendaal, C. et al., Clin. Exp. Immunol. 112:10-16 (1998)).

So far, only a few putative nuclear antigens of atypical p-ANCA have been suggested. Histone H1, and especially its isoform H1-3, has already been discussed as a candidate antigen for UC (Eggena, M. et al., J. Autoimmun. 14:83-97 (2000)). Also, 89% of the patients with AIH and atypical p-ANCA verifiably reacted to HMG 1 and 2 (high mobility non-histone chromosomal protein 1 and 2) (Sobajima, J. et al., Gut 44:867-873 (1999)). None of these nuclear proteins is specific for myeloid cells. However, an antigen of atypical p-ANCA suitable for assays must be detected by the majority of sera which contain atypical p-ANCA and be specific for myeloid cells.

For example, atypical p-ANCA can be found in the blood serum of patients with chronic inflammatory intestinal diseases (e.g. UC, rarely Crohn's disease) as well as with autoimmune diseases like, for example, AIH or PSC; atypical p-ANCA are already used in the clinical diagnostic procedures. The relatively complex indirect fluorescence microscopy is the most commonly used detection method. The use of highly specific solid phase assay will only be possible after the identification of the responsible antigen of atypical p-ANCA.

The subcellular localization of the target antigen of atypical p-ANCA in the nuclear envelope of myeloid cells (Terjung, B. et al., Hepatology 28:332-340 (1998)) and other work performed prior to the application resulted in the U.S. Pat. No. 6,627,458. It describes a nuclear envelope protein of neutrophils and myeloid cells as antigen of atypical p-ANCA which is detected by the majority (>92%, n=34/37) of the tested sera with atypical p-ANCA (Terjung, B. et al., Gastroenterology 119:310-322 (2000)). However, a reaction with atypical p-ANCA-negative sera of healthy test persons could not be detected. Neither did sera which contained classic p-ANCA show a reaction with this protein. Furthermore, the reaction could only be detected for myeloid differentiated cells (neutrophil granulocytes, human promyelomonocytic HL-60 cells, murine myelomonocytic 32D cells). The antigen in U.S. Pat. No. 6,627,458 was isolated from nuclear extracts of human neutrophils, HL-60 cells and murine 32D myeloid cells and basically characterized on the basis of its biochemical properties with the help of the SDS gel electrophoresis and isoelectric focusing. According to this, the described antigen had an apparent molecular weight (MW) of 50 kD and an isoelectric point (IEP, pI) of pH 6.0. Subsequent

tests performed by the inventors of the present application to identify the reactive protein by means of mass spectrometric techniques after having been purified by the gel electrophoresis were not successful. It appeared that the “reactive band” which could be detected in the one-dimensional gel electrophoresis presented a complex mixture of different proteins with the same molecular weight but with probably different isoelectric points. The mass spectrometric analyses resulted, among other things, in an evidence of the “beta chain of a mitochondrial ATP synthase” (52 kD, pI 5.0). Furthermore, the mass spectrometric analyses generally only detected one peptide which showed a correlation with a “diphosphooligosaccharide glycosyltransferase” (48 kD, pI 5.42), in another analysis with the “hepatocyte nuclear factor gamma” (47 kD, pI 8.29), the “heatshock protein 60” (58 kD, pI 5.23) or “apolipoprotein A1” (23 kD, pI 5.4) (each one with a low probability based Mowse score of 60-80; minimal required score to consider a detected protein as possible candidate protein: ≧46). All in all, four mass spectrometric analyses of the reactive protein from one-dimensional gels have been sampled (Protein Chemistry Core Facility, Columbia University, New York; Skirball Institute of Biomolecular Medicine, NYU, New York). The repeated identification of cytoplasmic proteins as possible candidate antigens of the atypical p-ANCA can most likely be attributed to a non-avoidable contamination of the nuclear envelope extracts with attached cytoplasmic parts. Despite complex purifying procedures, no purer fraction of the nuclear envelope proteins could be produced.

So far, no highly specific, reliable solid phase assays for the detection of atypical p-ANCA could be developed. Only the indirect immunofluorescence microscopy was available as detection method. However, the results received with this technique are always affected by the subjective assessment of the person who evaluates the immunofluorescence patterns. Indeed, some attempts have been made to develop ELISAS for the detection of atypical p-ANCA which considered the cytoplasmic proteins like the bactericidal/permeability increasing protein, cathepsin G, elastase or lactoferrin, rarely also myeloperoxidase (Zhao, M. H. et al., Clin. Exp. Immunol. 99:49-56 (1995); Halbwachs-Mecarelli, L et al., Clin. Exp. Immunol. 90:79-84 (1992); Peen, E. et al., Gut 34:56-62 (1993); Stoffel, M. P. et al., Clin.

Exp. Immunol. 104:54-59 (1996); Walmsley, R. S. et al., Gut 40:105-109 (1997)). However, these ELISAS only reacted with a minority of all sera positive for atypical p-ANCA (<35%). Thus, they are not appropriate for the use in the clinical diagnostics.

In view of the previously described research results, there was an urgent need of a reliable method for the detection of p-ANCA.

Microtubules and their principal constituents, tubulin alpha and tubulin beta, take part in a multitude of cellular processes in higher eukaryotes, among other things, the cell division, the cell motility and the preservation of the cell shape (Wang, D. et al., J. Cell. Biol. 1034:1903-1910 (1986); Lewis, S. A. et al., J. Cell. Biol. 101:852-861 (1985); Lewis, S. A. et al., Cell 49:529-548 (1987); Panda, D. et al., Proc. Natl. Acad. Sei. USA 91:11358-11362 (1994); Dumontet, C. et al., Cell Motil. Cytoskeleton 35:49-58 (1996)). Posttranslational modifications of the individual tubulin isotypes like, for example, the phosphorylation, the acetylation, the glycylation and the palmitoylation play an important role in the exercise of this functional specificity. Tubulin beta and tubulin alpha are generally considered as cytosolic proteins having the main task of forming microtubules. Tubulin beta is a ubiquitous protein which is existent in almost all cells. There are at least 7 different known isotypes (Roach, M. C. et al., Cell Motil Cytoskeleton 39:273-285 (1998); Wang, D. et al., J. Cell. Biol. 1034:1903-1910 (1986); Lewis, S. A. et al., J. Cell. Biol. 101:852-861 (1985)). Myeloid cell-specific isotypes are not included in these types. Although more than 800 tubulin genes (partly pseudo genes) are known (Lee, M. G. et al., Cell 33:477-487 (1983); Cleveland, D. W. und Sullivan, K. F., Ann. Rev. Biochem. 54:331-365 (1986)), no sequence information about myeloid cell-specific tubulin beta genes can be found in the relevant databases. A cross reactivity of atypical p-ANCA towards all so far identified tubulin isoforms is not known so far.

### SUMMARY OF THE INVENTION

Surprisingly, it could now be detected that the majority of the sera (>94%) of patients with chronic inflammatory intestinal diseases (like, for example, Crohn's disease) and autoimmune liver diseases (like, for example, AIH or PSC) which contain atypical p-ANCA reacted with a 50 kD, acid (pH value approx. 4.7-5.1) nuclear envelope protein which has been described here for the first time. This protein is specific for myeloid differentiated cells and has not yet been identified by means of sequence data as tubulin beta isotype 5 (TBB-5) or as a closely related isotype of tubulin beta which has not yet been captured in the databases.

Furthermore, it could surprisingly be detected that also TBB-5 itself (isolated from myeloid differentiated cells or recombinantly produced) as well as truncated and elongated forms of TBB-5 will be detected by atypical p-ANCA.

Furthermore, it could be surprisingly detected that the recombinant bacterial cell division protein FtsZ shows a cross reactivity with sera containing atypical p-ANCA. Tubulin beta isotype 5 shows a high structural homology with FtsZ, mainly in the area of the functionally important GTP binding site (FIG. 8). However, the amino acid sequences of both proteins (SEQ ID NO:2, SEQ ID NO: 17) are only homologous for 20%. This cross reactivity is also very interesting since a molecular mimicry between bacterial proteins of the intestinal bacteria and auto antigens of auto antibodies is consistently assumed for autoimmune liver diseases and the chronic inflammatory intestinal diseases (Burns, R., Nature 391:121-122 (1998); Selmi, C. et al., Hepatology 38:1250-1257 (2003); Cohavy, O. et al., Infect. Immun. 68: 1542-1548 (2000)).

Since atypical p-ANCA have a highly diagnostic precision for the above mentioned diseases (especially for the autoimmune liver diseases), the identification of antigens presents a decisive step for the completion of the diagnostic armentarium of these diseases. It could be surprisingly detected that atypical p-ANCA are the only relevant seromarkers for primary sclerosing cholangitis (PSC) as well as the seromarkers with the highest diagnostic precision for AIH.

The antigen of atypical p-ANCA according to this invention can be recombinantly produced. This means that a complex preparation of the nuclear envelope for the extraction of the antigen will not be required.

The antigen of atypical p-ANCA according to this invention can be used for the preparation of highly specific assays (especially solid phase assays) for the detection of atypical p-ANCA since it is an isolated protein. For the first time, the identification of this antigen allows the development of such p-ANCA-specific solid phase assays which presents a significant improvement and especially a significant simplification in comparison with the so far usual diagnostic methods.

The invention thus relates to

(1) a method for the identification and/or quantification of atypical anti-neutrophil cytoplasmic antibodies (hereinafter referred to as “atypical p-ANCA”) in a sample by means of an isolated antigen which is able to bind with atypical p-ANCA (hereinafter referred to as “atypical p-ANCA antigen”);

(2) a preferred embodiment of method (1), where the antigen is a protein with a molecular weight of approx. 50 kD, determined by the SDS-PAGE gel electrophoresis (using commercial molecular weight markers) and an isoelectric point (IEP) of pH 4.7-5.1, determined by isoelectric focusing (using commercial IEP standards);

(3) a preferred embodiment of method (1) or (2), where the antigen

(i) can be isolated from tubulin preparations of human or vertebrate cells, preferably from blood cells, cells of the immune system, hepatocytes, cells of the bile system or cells of the intestinal mucosa, especially from neutrophil granulocytes, and/or

(ii) can be isolated from nuclear envelope preparations of myeloid differentiated cells;

(4) a preferred embodiment of method (1), (2), or (3), where the antigen

(i) is a tubulin beta, especially a TBB-5 with the amino acid sequence SEQ ID NO:2 or a substitution mutant, a deletion mutant and/or an addition mutant of the same, and/or

(ii) is a bacterial cell division protein, especially the bacterial cell division protein FtsZ with the amino acid sequence SEQ ID NO:17, or a substitution mutant, a deletion mutant and/or an addition mutant of the same;

(5) a preferred embodiment of method (1) to (4) which includes the quantitative determination of the concentration of atypical p-ANCA in a sample by means of an immunoassay and/or a comparison with a standard concentration of atypical p-ANCA;

(6) a protein as defined in (1) to (4), but not TBB-5 with the amino acid sequence SEQ ID NO:2 or FtsZ with the amino acid sequence SEQ ID NO:17;

(7) a DNA which encodes for a protein according to (6);

(8) a vector which includes a DNA according to (7);

(9) a host organism which transforms with a vector according to (8) or is transfected and/or has a DNA according to (7);

(10) a method for the production of a protein according to (6) including the cultivation of the host organism according to (9);

(11) a kit for the identification and/or quantification of atypical p-ANCA according to (1) to (5), preferably containing an antigen as defined in (1) to (4) and/or a microbial strain of a cell line which is appropriate to express this antigen (preferably recombinantly); and

(12) the use of the protein defined in (1) to (4) or (6) in immunological, neurobiological and cell physiological analyses, in the clinical research, as well as for diagnostic methods in vivo and in vitro.

Due to the high purity of the antigen, the method (1) according to the invention does not result or only rarely results in unspecific cross reactions.

### BRIEF DESCRIPTION OF THE FIGURES

FIG. 1: Reactivity of atypical p-ANCA with a 50 kD nuclear envelope protein and “fingerprinting” of the p-ANCA antigens. Separation of nuclear envelope extracts of HL-60 cells by means of a two-dimensional gel electrophoresis, followed by Western Blotting, immunodetection with sera containing “atypical” p-ANCA, and detection of reactive spots by means of chemiluminescence. Gel strips with immobilized pH gradients (pH value 4.7-5.9) were used for the procedure of isoelectric focusing.

(A) Detection of 2-3 beadlike reactive spots with a molecular weight of 50 kD and a pH value of 4.9-5.1 in the gel stained with silver nitrate (ellipsis).

(B) During the immunodetection with sera containing “atypical” p-ANCA (serum of a patient with autoimmune hepatitis and atypical p-ANCA, serum endpoint titre 1:2,560), reactive spots (ellipsis) could be determined in the same pH value range. The spots detected in the gel could definitely be attributed to the spots reacting in the immunoblot.

The molecular mass in kD is specified on the left side of the gel and the immunoblot. For the immunodetection, the sera were diluted at a ratio of 1:500.

(C) The MALDI-TOF-MS analysis of the reactive spots resulted in fragments which were homologous with sequence sections of tubulin beta isotype 5 (TBB-5; Swiss-Prot P05218). Since both reactive spots were identified independently as homologous with TBB-5 but showed slightly different molecular masses, posttranslational modifications which will result in these slightly different pH values are very likely.

FIG. 2: Reactivity of atypical p-ANCA with proteins from tubulin preparations of HL-60 cells. The tubulin preparations from HL-60 cells were separated by a one-dimensional or a two-dimensional gel electrophoresis. Gel strips with immobilized pH gradients (pH value 4.7-5.9) were used for the procedure of isoelectric focusing. The immunodetection was used for the visualization of tubulins reacting with atypical p-ANCA.

(A) After the one-dimensional (ID) gel electrophoresis, the predominant majority of the sera containing atypical p-ANCA (94%; 32/34) reacted with a tubulin showing a molecular weight of 50 kD (line 1: serum of a patient with autoimmune hepatitis [AIH], titer of the atypical p-ANCA 1:5,120; line 2: serum of a patient with primary sclerosing cholangitis [PSC], titer of the atypical p-ANCA 1:320; line 3: serum of a patient with AIH and ulcerative colitis [UC], titer of the atypical p-ANCA 1:1,280). No reactivity with one of the electrophoretically separated proteins could be detected in sera of healthy control persons (line 4).

(B) An immunoblotting with tubulin extracts showed two protein spots with a molecular weight of 50 kD and pI values between 4.9-5.1 reacting with serum of patients containing atypical p-ANCA. The incubation was accomplished with a serum containing atypical p-ANCA (see also (A), line 1). The molecular weight in kD is specified on the left side of the gel and the immunoblot. For the immunodetection, the sera were diluted at a ratio of 1:500.

(C) A MALDI-TOF mass spectrometric analysis of both reactive spots from FIG. 2B with a subsequent search in the protein database (Swiss-Prot; MASCOT) confirmed tubulin beta isotype 5 (TBB-5) as the target antigen of atypical p-ANCA. Specified are the amino acid sequence of TBB-5 (444 amino acids; SEQ ID NO:2) as well as the peptides determined in the mass spectrometric analysis (44% of the entire amino acid sequence; blue bold print). The identified peptides have the following positions: 1-19, 47-58, 63-78, 104-122, 155-162, 163-174, 217-241, 242-252, 253-262, 263-276, 283-297, 310-318, 363-377, 381-391. A “signal peptide” (highlighted in grey color) could be identified at the position 283-297 allowing the clear assignment to TBB-5.

FIG. 3: Atypical p-ANCA detect TBB-5 but not TBB-1. Tubulin extracts from HL-60 cells were separated by means of a two-dimensional gel electrophoresis.

(a) A reactive spot with a molecular weight of approx. 50 kD and a pI value of approx. 4.95 was detected with antibodies to tubulin beta isotype 1 (diluted at a ratio of 1:20,000).

(b) The incubation of the same immunoblot with a serum containing atypical p-ANCA resulted in two reactive spots with a molecular weight of approx. 50 kD but showing a slightly increased pI value (approx. 5.0) compared to TBB-1.

(c) The computer simulation (Adobe Photoshop, version 6.0 (San Jose, Calif.)) shows a virtual overlap of both figures (a) and (b) and confirms that anti-TBB-1 and atypical p-ANCA detect different proteins with the same molecular weight but with slightly different pI values.

The molecular weight in kD is specified on the left side of the immunoblot. For the immunodetection, the sera were diluted at a ratio of 1:500.

FIG. 4: Myeloid cell-specific reactivity of atypical p-ANCA. Electrophoretic separation of tubulin preparations from human promyelomonocytic HL-60 cells, human HeLa cells and human HepG2 cells and subsequent immunodetection with sera containing atypical p-ANCA. Here, only a reaction with a 50 kD protein from electrophoretically separated tubulin preparations of HL-60 cells (line 1, 2) could be detected. No reaction could be detected for HeLa cells (line 3, 4) or HepG2 cells (line 5, 6). Lines 1, 3, and 5: Incubation with a serum containing atypical p-ANCA of a patient suffering from autoimmune hepatitis (1:1,280). Lines 2, 4, and 6: Serum of a patient with primary sclerosing cholangitis and atypical p-ANCA (1:320).

FIG. 5: Microscopic immunofluorescence pattern of atypical p-ANCA before and after the affinity purification and preabsorption. Synthesis of the staining patterns with conventional indirect immunofluorescence microscopy of ethanol-fixed neutrophil granulocytes. Atypical p-ANCA were detected by secondary antibodies marked with fluorescein isothiocyanate.

(A) Ethanol-fixed neutrophil granulocytes were incubated with the serum of a patient with autoimmune hepatitis containing atypical p-ANCA (serum titer 1:640). The characteristic fluorescence pattern of atypical p-ANCA showing an annular staining pattern of the nuclear periphery and an intranuclear mottled pattern can be detected.

(B) To obtain affinity-purified atypical p-ANCA, tubulin was separated by means of a one-dimensional SDS-PAGE. Then, the proteins were transferred to a nitrocellulose membrane, and a reactive band with a molecular weight of approx. 50 kD was immunodetected by the incubation with a serum containing atypical p-ANCA. This reactive band was cut out from the nitrocellulose membrane. Bound atypical p-ANCA were eluted with an acid solution from 200 mM of glycine (pH value 2.8) and 1 mM EDTA and used for the immunofluorescence microscopy. The obtained staining pattern shows the same typical characteristics of atypical p-ANCA as could be detected before the affinity purification (compare to (A)).

(C) Fluorescence patterns of atypical p-ANCA from the serum of a patient with ulcerative colitis (1:640).

(D) Serum from (C) was preabsorbed with a tubulin preparation at a ratio of 1:10. After this, no atypical p-ANCA could be detected in the fluorescence microscopy.

Bar length: 10 μm. Sera which were not affinity-purified and not preabsorbed were diluted at a ratio of 1:10 whilst affinity-purified and preabsorbed sera were undilutedly used.

FIG. 6:

(A) Transient transfection of Cos-7 cells with cDNA plasmids encoding for TBB-5, and subsequent electrophoretic separation of the cell lysates. An additional Xpress epitope was integrated at the N-terminus of the respective cDNA for the specific detection. After the immunodetection, the expressed fusion proteins were detected with anti-Xpress antibodies (diluted at a ratio of 1:1,000) by means of a chemiluminescence reaction.

A clear reactive band could be shown for the expressed full-length TBB-5 (BC007605, line 3) and a TBB-5 elongated with GFP (GFP-BC007605, line 5) as well as for two truncated proteins including the N-terminus or the C-terminus of TBB-5 (line 1: BB, line 2: BP). On the other hand, only one slightly reactive band was detected for the truncated fusion protein BE which includes a shorter section of the N-terminus of TBB-5 compared to BB (line 4). The double band for BP suggests

proteolytic decomposition products. See FIG. 6B for the structure of the vector constructs.

(B) Vector constructs for the production of truncated TBB-5 or GFP TBB-5 fusion protein. BC007605, full-length TBB-5 (theoretical molecular weight 55 kD). BE, 32 kD. BB, 45 kD. BP, 30 kD. BC007605-GFP, 81 kD.

FIG. 7: Reactivity of recombinantly produced full-length TBB-5, GFP TBB-5 as well as truncated fragments of TBB-5 with different sera. Recombinantly produced full-length TBB-5, GFP TBB-5 and truncated fragments of TBB-5 (BB, BP) were separated by means of the one-dimensional gel electrophoresis (see also FIG. 6) and, as described in example no. 5, tested with sera containing atypical p-ANCA or sera which did not contain atypical p-ANCA of healthy control persons. Reactive protein bands were detected by means of the immunodetection and a subsequent chemiluminescence reaction (see also example no. 5). Highly specific and highly sensitive antibodies which are directed to the integrated Xpress epitope of the expressed fusion proteins (diluted at a ratio of 1:1,000) were used in the immunodetection procedure for testing the respective qualitative and quantitative protein expression and reactivity.

(A) A clear reactivity of the expressed full-length TBB-5 (approximate molecular weight: 55 kD) with the anti-Xpress antibody (lines 1 and 8) as well as with sera of patients with autoimmune hepatitis and sera containing atypical p-ANCA (lines 2-5) could be detected. On the other hand, control sera not containing atypical p-ANCA showed no reactivity with the expressed full-length TBB-5 (lines 6, 7, and 9).

(B) The expressed truncated fragment BB was detected by the anti-Xpress antibody (line 11) as well as by sera containing atypical p-ANCA (line 10) as reactive protein band with a molecular weight of approx. 45 kD. The same patient serum was used as in line 5.

(C) and (D) Additionally, specific reactivities with the truncated fragment BP (approximate molecular weight: 30 kD, double band possibly referred to the proteolytic decomposition of fragment BP) and, when using the expressed GFP TBB-5, with anti-Xpress antibodies (lines 13 and 15) as well as when using sera containing atypical p-ANCA (lines 12 and 14) were detected. The same serum as for the immunodetection in line 2 was used. Since the truncated fragment BE could only be expressed in

too small amounts, continuous tests with sera for specific reactivities were not performed.

FIG. 8: Comparison of FtsZ from E. coli with human tubulins. Three-dimensional reconstruction of FtsZ from E. coli, human tubulin beta and tubulin alpha. All three molecules show a highly structural homology, especially in the area of the GTP binding site (highlighted in green color) with a match of the amino acid sequence of not more than 20%. Matches of the α-helices are marked in orange color, whereas matches of the β chain are marked in purple. Areas which differ from each other (especially in the area of the C-terminus of the three molecules) are highlighted in grey color. Modified according to Burns, R., Nature 391:121-122 (1998).

FIG. 9: Reactivity of FtsZ with atypical p-ANCA. FtsZ recombinantly produced in E. coli was separated by means of a two-dimensional gel electrophoresis and, in the gel stained with Coomassie (A), showed a characteristic spot with an approximate molecular weight of 42 kD and an isoelectric point of 4.8. A clear reactivity of the protein with atypical p-ANCA could be detected in the immunoblot (B) (see also example no. 15). Mass spectrometric analyses of the reactive spot confirmed this spot as FtsZ.

Sequence listing free text

| | |
|---|---|
| SEQ ID | |
| NO: | Free text |

| | |
|---|---|
| 1 | DNA TBB-5 BC007605 |
| 2 | Protein TBB-5 P05218 |
| 3 | “Signal peptide,” partial sequence of TBB-5 |
| 4 | TBB-5 fragment BE |
| 5 | TBB-5 fragment BE |
| 6 | TBB-5 fragment BB |
| 7 | TBB-5 fragment BB |
| 8 | TBB-5 fragment BP |
| 9 | TBB-5 fragment BP |
| 10 | Linker primer 3′ end, EcoRI site for cloning |
| | BP in pcDNA3.1 |
| 11 | Linker primer 3′ end, XhoI Site for cloning |
| | in pQETris |
| 12 | Linker primer 5′ end, KpnI Site for cloning |
| | BP in pcDNA3.1 |
| 13 | Linker primer 5′ end, KpnI Site for cloning |
| | in pQETris |
| 14 | Forward primer reverse transcription TBB-5 |
| 15 | Reverse primer reverse transcription TBB-5 |
| 16 | FtsZ |
| 17 | FtsZ |
| 18 | Linker primer 3′ end, XhoI Site for cloning |
| | FtsZ in pQETris |
| 19 | Linker primer 5′ end, NcoI Site for cloning |
| | FtsZ in pQETris |
| 20 | Partial sequence of the antigen of atypical |
| | p-ANCA |
| 21 | Partial sequence of the antigen of atypical |
| | p-ANCA |
| 22 | Partial sequence of the antigen of atypical |
| | p-ANCA |
| 23 | Partial sequence of the antigen of atypical |
| | p-ANCA |
| 24 | Partial sequence of the antigen of atypical |
| | p-ANCA |
| 25 | Partial sequence of the antigen of atypical |
| | p-ANCA |
| 26 | Partial sequence of the antigen of atypical |
| | p-ANCA |
| 27 | Partial sequence of the antigen of atypical |
| | p-ANCA |
| 28 | GFP full-length TBB-5 (BG) |
| 29 | GFP full-length TBB-5 (BG) |
| 30 | Linker primer forward (KpnI) for GFP TBB-5 |
| 31 | Linker primer backwards (EcoRI) for GFP |
| | TBB-5 |
| 32 | Linker primer forward (KpnI) for TBB-5-myc |
| | in pcDNA3.1 |
| 33 | Linker primer backwards (EcoRI) for |
| | TBB-5-myc in pcDNA3.1 |
| 34 | DNA full-length TBB-5 with c-myc tag |
| 35 | Full-length TBB-5 with c-myc tag |
| 36 | FtsZ peptide |

### DETAILED DESCRIPTION OF THE INVENTION

The present invention relates to the molecular characterization and identification of several antigens which will be detected by atypical p-ANCA. It could be proved that the majority of sera of patients with autoimmune liver diseases or ulcerative colitis reacted with an acid myeloid cell-specific 50 kD protein from nuclear envelope preparations that is homologous to TBB-5. When using tubulin preparations from myeloid differentiated cells, 94% of the sera reacted with a protein with a weight of 50 kD and a pI value of 4.9-5.1. Mass spectrometric data confirmed that this protein was TBB-5. Thus, the present invention allows an improved diagnosis of patients with chronic inflammatory intestinal diseases (like, for example, UC or Crohn's disease) and autoimmune liver diseases (like, for example, AIH or PSC).

In connection with the present invention, “atypical p-ANCA antigen” means a native antigen which will be detected and bound in vivo and in vitro by atypical p-ANCA, as well as those non-native antigens of atypical p-ANCA which will also be bound in vivo and in vitro with high sensitivity and specificity by atypical p-ANCA, especially proteins showing high sequential homologies to tubulin beta isoform 5, especially tubulin beta isoform 5 itself and its truncated and elongated mutants as well as bacterial FtsZ and its truncated and elongated mutants. An antigen of atypical p-ANCA in accordance with the invention can also especially be recombinantly produced.

A “fusion protein” in the context of the present invention includes at least one defined protein or peptide which is linked to a second protein or peptide (“marker protein”). The nucleotide sequences encoding for the individual parts of the fusion protein are linked in a way which allows the expression under the control of a single promoter.

A “marker protein” in terms of the present invention is preferably a non-toxic, directly or indirectly detectable protein or peptide, preferably either a functional protein (e.g. a fluorescence marker like GFP or an enzyme) or a peptide facilitating the detection and/or isolation of the fusion protein (e.g. an Xpress tag, a His tag, or a c-myc tag). In the case of a functional protein being a marker protein, this can preferably be detected by means of a calorimetric assay, e.g. with the Lowry method, with Coomassie blue according to Bradford, sulforhodamine B, β galactosidase (lacZ), placental alkaline phosphatase (PAP), fluorescence, bioluminescence like, for example, firefly luciferase, phosphorescence, chemiluminescence or similar detection methods (Haughland, R. P., Molecular Probes. Handbook of fluorescent probes and research Chemicals, 172-180, 221-229 (1992-1994); Freshney, R. I., Culture of animal cells, 3^{rd} ed., Wiley & Sons, Inc. (1994)). The use of a fluorescent protein or peptide is preferred. Especially preferred is the use of GFP and its mutants as well as of “Reef Coral Fluorescent Proteins” (RCFP) like, for example; AmCyan, ZsGreen, ZsYellow, DsRed, AsRed and HcRed (Clontech, Palo Alto, Calif.). Especially preferred are GFP and its mutants (overview see Labas, Y. A. et al., PNAS 99(7):4256-4261 (2002)). Very especially preferred is GFP.

In the case of a protein, “isolated” means that is has been separated or purified from other proteins with which it is normally associated in the organism in which it can naturally be found. This includes biochemically purified proteins, recombinantly produced proteins and chemically synthesized proteins. This definition can also be applied to nucleic acids, especially DNA, and peptides.

“Native” will be used in the same sense as “natural (naturally occurring)”.

In the context of the present invention, nucleic acid sequences, especially DNA sequences coding for the proteins or peptides according to the invention, are either identical or substantially identical with the native sequence or the underlying artificial sequence according to the invention. If a special nucleotide sequence is mentioned in the context of this invention, this sequence itself as well as its substantially identical sequences will be included. “Substantially identical” in this context means that only an exchange of bases has occurred in the sequence in the scope of the degenerated nucleic acid code. This in turn means that the codons within encoded sequences of

the substantially identical nucleic acid are only changed in comparison to the original molecule in a way which does not result in a modification of the amino acid sequence of the translation product (normally an exchange of the codon by another codon of its codon family). In the scope of the present invention, especially those sequences which are specified in the sequence listing will be preferred.

In the scope of the present invention, protein sequences and peptide sequences can be modified by the substitution of amino acids. Such substitutions are preferred which preserve the functionality and/or the conformation of the protein or the peptide. Especially preferred are those substitutions in which one or more amino acids are replaced by amino acids with similar chemical properties, e.g. valin by alanin (“conservative amino acid substitution”). The proportion of the substituted amino acids compared to the proportion of the native protein or, if it is not a native protein, compared to the initial sequence is preferably 0-30% (related to the number of amino acids in the sequence); especially preferred is a proportion of 0-15%, and very especially preferred is a proportion of 0-5%.

Nucleic acid sequences and amino acid sequences can be used as full-length sequences or as addition or deletion products of these full-length sequences for the execution of the invention. In terms of the amino acid sequences, the addition products also include fusion proteins as well as amino acid sequences which are generated by the addition of 1-200 amino acids, or preferably 1-50 amino acids; very especially preferred is the addition of 1-20 amino acids. The added amino acids can be added or attached individually or in continuous sections of 2 or more linked amino acids. The addition can take place at the N-terminus and/or the C-terminus and/or within the original sequence. Several additions in one sequence are admissible, whereas a single addition is preferred. Especially preferred is an addition at the C-terminus or the N-terminus.

If not otherwise specified for special sequences, the deletion products of the full-length amino acid sequences are produced by the deletion of 1-220 amino acids; preferred is the deletion of 1-100 amino acids, very especially preferred is the deletion of 1-50 amino acids. The deleted amino acids can be removed individually or in continuous sections of 2 or more connected amino acids. The deletion can take place at the N-terminus and/or the C-terminus and/or within the

original sequence. Several deletions in one sequence are admissible, whereas a single deletion is preferred. Especially preferred is a deletion at the C-terminus or the N-terminus. The admissible deletions and additions in the nucleic acid sequences according to the invention have been made in the scope and to the extent which corresponds to the admissible amino acid deletions or amino acid additions. The addition or deletion of individual or paired bases is possible in addition to the deletion and addition of entire codons.

A fragment of a nucleic acid or of a protein is a part of its sequence which is shorter than its full length but which contains a sequence section which is the minimum requirement for the hybridization or the specific bond. In the case of a nucleic acid, this sequence section is still able to hybridize with the native nucleic acid under stringent conditions and preferably includes at least 15 nucleotides; especially preferred are at least 25 nucleotides. In the case of a peptide, this sequence section is sufficient to allow a bond of an antibody specific for the native protein.

The term “truncated” specifies a shortened amino acid sequence or nucleic acid sequence, whereas the term “elongated” specifies an elongated amino acid sequence or nucleic acid sequence.

A preferred characterization of the natural atypical p-ANCA antigen according to the invention and according to embodiments (1)-(3) includes the production of nuclear extracts from myeloid differentiated cells, preferably from human neutrophil granulocytes, human HL-60 cells and murine 32D myeloid cells. The isolated proteins will be separated by means of a one-dimensional and two-dimensional gel electrophoresis. Reactive proteins will be identified by immunoblotting with sera containing atypical p-ANCA and the respective negative controls without atypical p-ANCA. The antigen which will be detected by atypical p-ANCA will be purified and biochemically characterized as well as characterized with respect to its amino acid sequence.

Based on the test results presented in U.S. Pat. No. 6,627,458, the two-dimensional gel electrophoresis has been optimized for the present invention. In contrast to the previous method, nuclear envelope extracts were further separated by means of immobilized pH gradients (see also example no. 6). With this method, well reproducible

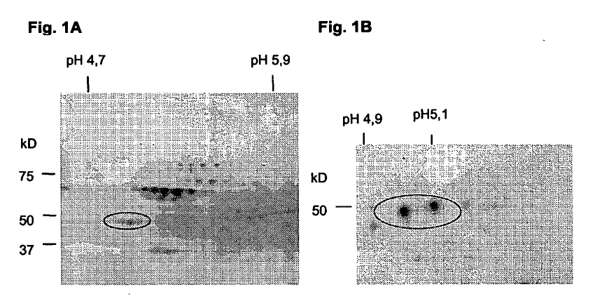

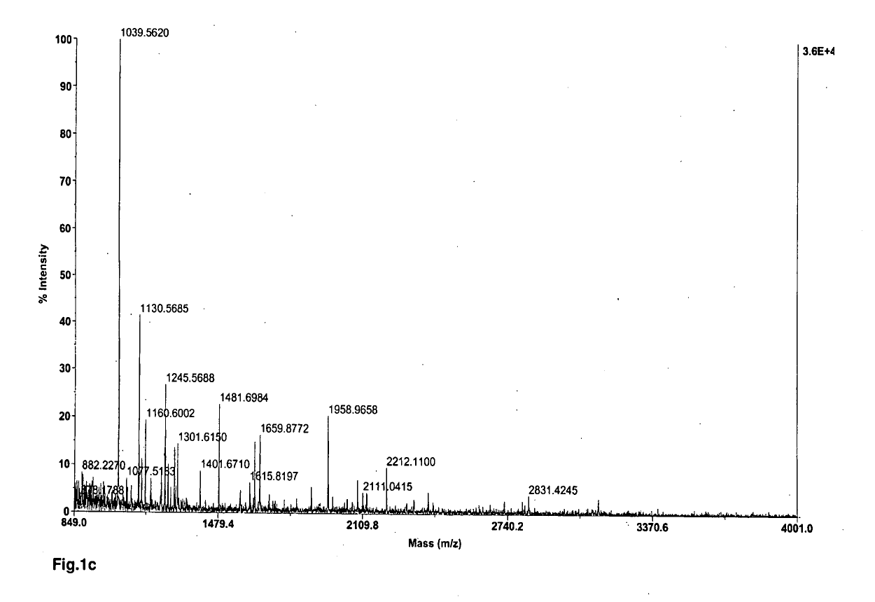

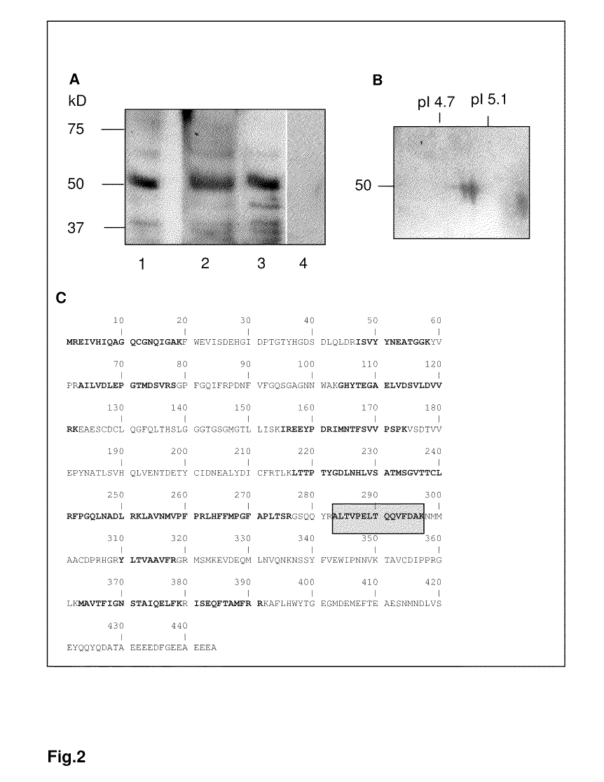

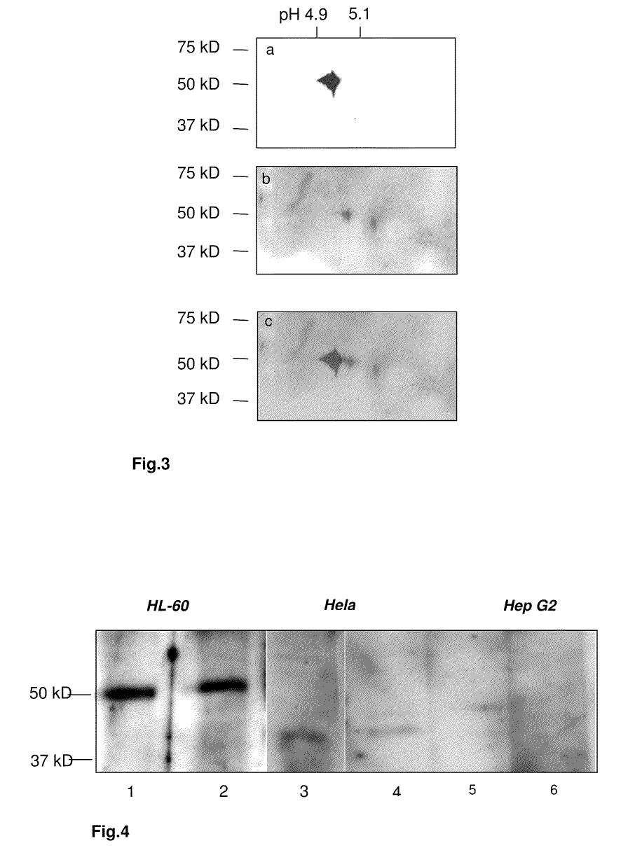

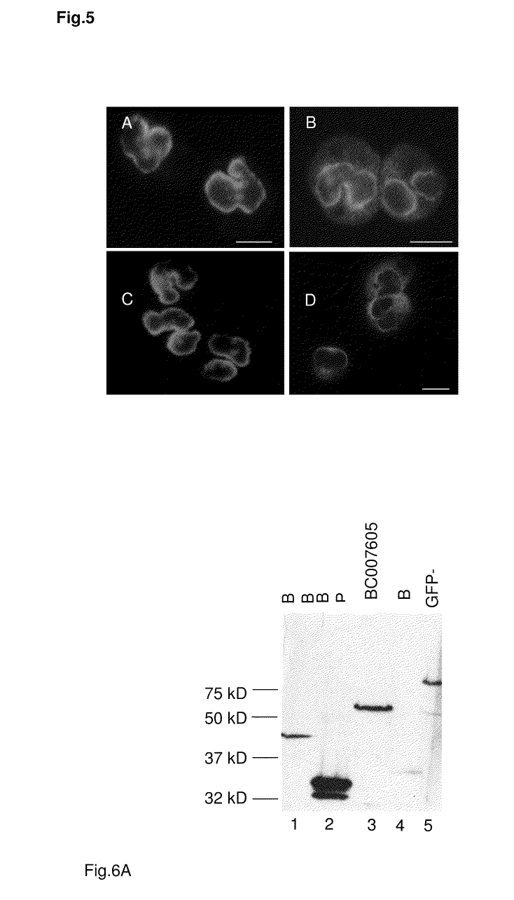

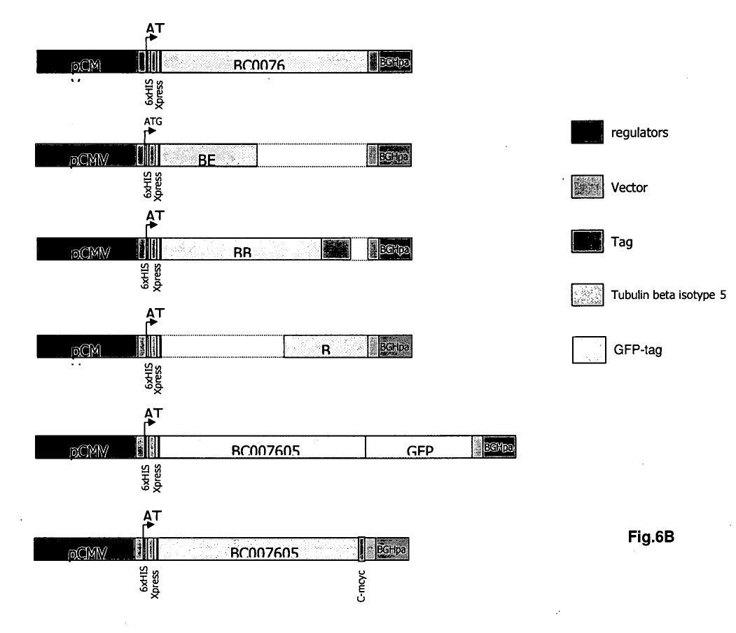

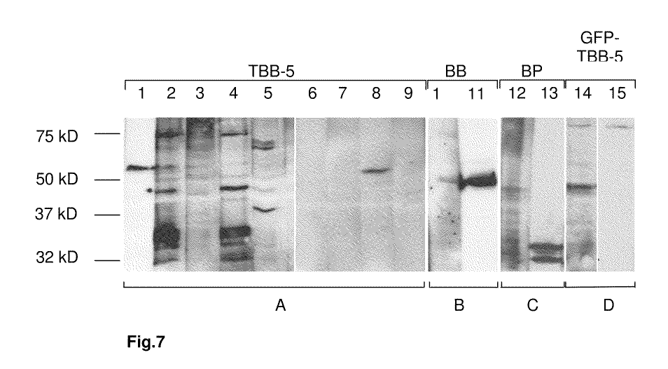

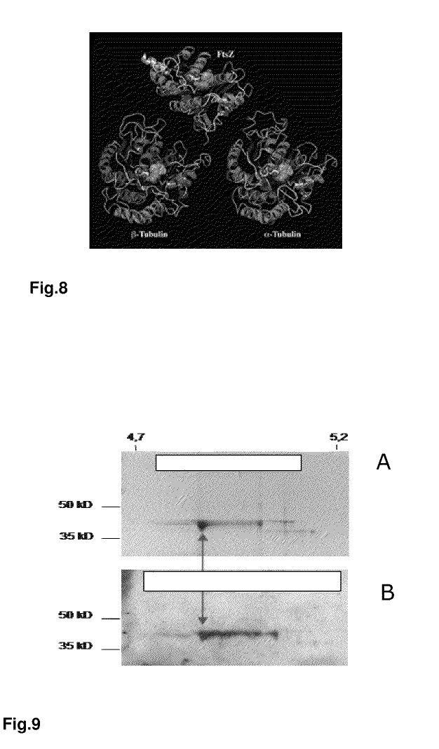

results could be achieved. By the method of immunoblotting, two “spots” with an approximate molecular weight of 50 kD and an isoelectric point between 4.9 and 5.1 could be identified on a regular basis (FIG. 1), these spots were also detected by the majority (>94%) of the tested sera of patients with PSC or AIH containing atypical p-ANCA (32 of 34 tested sera with atypical p-ANCA, thereof AIH: 26/27 and PSC 6/7; see example no. 6). None of the sera of ANCA-negative healthy individuals (n=5) reacted with these 50 kD proteins. Nor did sera of patients with AIH or PSC and classic p-ANCA (n=3) detect the 50 kD protein.

With the help of mass spectrometric analyses (e.g. MALDI-TOF) of these reactive spots in the immunoblot (FIG. 1C), it was possible (after accessing protein databases (e.g. Swiss-Prot, MASCOT)) to assign the detected peptide masses with a high probability (Mowse based Probability score 318-780; required minimum score for the protein identification: ≧46) to tubulin beta isotype 5 (TBB-5) (Swiss-Prot: P05218). These findings were reproducible, especially in different mass spectrometry laboratories (ZMMK, University of Cologne (Germany), Swiss-Prot, Geneva, Switzerland). Interestingly, both reactive spots were independently identified as TBB-5. This increases the probability of posttranslational modifications of the protein that result in slightly different pI values.

Tubulin preparations from HL-60 cells were produced for the further confirmation of the result saying that TBB-5 is the target antigen of atypical p-ANCA (see also example no. 4). These preparations contained mixtures of different tubulins and tubulin-binding proteins. After the protein-chemical separation of the tubulin fractions with the help of the one-dimensional or the two-dimensional gel electrophoresis, a reactivity with a 50 kD acid (pI 4.9-5.1) protein could be detected again for 94% (32/34; AIH: 26/27, PSC: 6/7) of the sera containing atypical p-ANCA (FIGS. 2A and 2B). Sera which did not contain atypical p-ANCA or sera with classic p-ANCA did not detect any of the electrophoretically separated tubulins. Mass spectrometric analyses in two independent laboratories definitely confirmed tubulin beta isotype 5 as the protein reacting with sera containing atypical p-ANCA (FIG. 2C). However, the identified peptides only included 44-50% of the amino acid sequence of TBB-5 (as in the previous mass spectrometric analyses). In the area of the entire C-terminus of the amino acid sequence (amino acids

391-444, total number of amino acids: 444) no peptides could repeatedly be sequenced. This is important since significant differences of the amino acid sequence between the known human highly homologous tubulin beta isotypes 1-5 (>95%) are predominant mainly in this area. However, the protein TBB-5 reacting with sera containing atypical p-ANCA could be definitely differentiated from the other, strongly homologous isotypes of tubulin beta by the exact identification of the amino acid sequence of the peptide at the position amino acid 283-297 (“signal peptide” with SEQ ID NO:3, according to sequence ALTVPELTQQVFDAK). Thus, an assignment to TBB-5 is again very likely (FIG. 2C). However, it cannot be ultimately excluded that the protein to be defined is a special isotype of tubulin beta showing a high homology with TBB-5 but whose amino acid sequence differs from the database entries (P05218 Swiss-Prot, NCBI) in the so far not identified areas. Furthermore, it may be important that both reactive spots closely adjoining in the two-dimensional gel electrophoresis were individually analyzed by mass spectrometry, and both detected peptide sequences independently corresponded to TBB-5. Both detected spots reacting with atypical p-ANCA can probably be explained by posttranslational modifications of the TBB-5 protein which result in isoelectric values of TBB-5 which do not differ enough from each other. Some signs for a relevant phosphorylation of the protein are existent.

Additionally, the reactivity of atypical p-ANCA has been shown with TBB-5 but not with tubulin beta isotype 1 (TBB-1): The two-dimensional gel electrophoresis resulted in closely adjoining but clearly distinguishable reactive spots during the incubation of the immunoblots either with antibodies to TBB-1 or with atypical p-ANCA (FIG. 3). Although the amino acid sequences of TBB-1 and TBB-5 are almost identical and both TBB isotypes of HL-60 cells are expressed in large amounts, atypical p-ANCA only reacted with TBB-5. Since the used antibody to TBB-1 has been produced to the carboxy terminus of TBB-1 which is identical for TBB-1 and TBB-5 with regard to the amino acid sequence basis, posttranslational modifications seem to be responsible for this differentiation.

Furthermore, the myeloid cell-specific reactivity of tubulin beta isotype 5 with atypical p-ANCA has been confirmed in the scope of the present invention.

This is an important prerequisite that a protein can act as potentially useable target antigen of atypical p-ANCA in the clinical diagnostics. As becomes obvious in the term “anti-neutrophil cytoplasmic antibodies”, atypical p-ANCA react with an antigen which is specific for neutrophil granulocytes or myeloid differentiated cells. However, it remains unclear if the protein to be defined is exclusively expressed in these cells and/or if the expression in the other cells of the respective organism has decreased. To detect the myeloid cell-specific reactivity of TBB-5 with atypical p-ANCA, tubulin fractions from different non-myeloid cells were produced, and a respective reactivity was tested by means of the immunoblot method. Here, no significant reactivity with tubulin preparations from non-myeloid differentiated cells (e.g. HeLa cells, HepG2 cells, Cos 7 cells or NIH3T3 cells) could be detected (FIG. 4). In contrast, a clear reaction of atypical p-ANCA could be determined with tubulin preparations from myeloid differentiated cells (here: HL-60 cells). Although the reactivity of the atypical p-ANCA was mainly directed against the 50 kD protein in tubulin extracts from HL-60 cells, 15% (n=5/34) or 6% (n=2/34) of the sera containing atypical p-ANCA also reacted with proteins of the tubulin preparations from HeLa cells or HepG2 cells. Except for one serum, the antigen here was a protein with an apparent molecular weight of 48 kD and was identified by MS Fingerprinting as being a complex mixture of different proteins which are not related to TBB-5 or other tubulin isotypes. Sera of healthy individuals did not show a specific reactivity with the different tubulin preparations.

The identification of TBB-5 as target antigen of atypical p-ANCA is very surprising because tubulin beta is existent in almost all cells and is not a myeloid cell-specific protein (Dumontet, C. et al., Cell Motil Cytoskeleton 35:49-58 (1996); Lewis, S. A. et al., J Cell Biol 101:852-861 (1985); Roach, M. C. et al., Cell Motil Cytoskeleton 39:273-285 (1998); Wang, D. et al., J. Cell Biol. 1034:1903-1910 (1986)).

Also the fluorescence patterns of atypical p-ANCA after the affinity purification procedure or after the preabsorption with tubulin extracts confirmed the reaction of atypical p-ANCA with tubulins:

Representative sera containing atypical p-ANCA (PSC n=3, AIH n=5) which have been affinity-purified by a bond to the protein homologous to TBB-5 from the tubulin extracts of HI-60 cells showed the same characteristic fluorescence pattern for atypical p-ANCA

on ethanol-fixed granulocytes like atypical p-ANCA which have not been affinity-purified (FIGS. 5A and 5B). If, on the other hand, sera containing atypical p-ANCA were excessively preabsorbed with tubulin extracts from HL-60 cells, the fluorescence pattern of atypical p-ANCA existent prior to the preabsorption procedure could not be detected anymore (FIGS. 5C and 5D).

The atypical p-ANCA antigen isolated from nuclear envelope preparations according to embodiment (2) of the invention has an apparent molecule weight of approx. 50 kD (estimated according to an SDS gel electrophoresis by comparison with molecular weight markers) and an isoelectric point of approx. pH 4.7-5.1 (estimated according to isoelectric focusing through two-dimensional gel electrophoresis by comparison with markers for isoelectric focusing). This target antigen will be detected by more than 94% of the tested patient sera. This exceeds the value mentioned in U.S. Pat. No. 6,627,458.

The natural antigen of atypical p-ANCA isolated from nuclear envelope preparations according to embodiment (1), (2) or (3) preferably includes the partial sequence SEQ ID NO:3 and/or one or more of the partial sequences selected from SEQ ID NO:20, SEQ ID NO:21, SEQ ID NO:22, SEQ ID NO:23, SEQ ID NO:24, SEQ ID NO:25, SEQ ID NO:26 and SEQ ID NO:27; especially preferred are SEQ ID NO:20, SEQ ID NO:21, SEQ ID NO:22 and SEQ ID NO:23. These parts of the protein sequence of the atypical p-ANCA antigen were determined by mass spectrometric methods. Object of the invention are also nucleic acids which include nucleic acid fragments encoding for such proteins, preferably DNA sequences and cDNA.

The sequential comparison with known protein sequences confirmed that tubulin beta isoform 5 shows a high homology with the atypical p-ANCA antigen on a sequential level (FIG. 2C).

The antigen of atypical p-ANCA according to embodiment (1), (2) or (3) thus includes in a preferred embodiment the partial sequence SEQ ID NO:3 and/or one or more of the partial sequences selected from SEQ ID NO:20, SEQ ID NO:21, SEQ ID NO:22, SEQ ID NO:23, SEQ ID NO:24, SEQ ID NO:25, SEQ ID NO:26 und SEQ ID NO:27; especially preferred are SEQ ID NO:20, SEQ ID NO:21, SEQ ID NO:22 und SEQ ID NO:23. Objects of the invention are also those nucleic acids which include nucleic acid fragments encoding for such proteins, especially DNA sequences and cDNA.

In one aspect of embodiment (4), the antigen is TBB-5 (SEQ ID NO:2) and/or will be encoded by a DNA with SEQ ID NO:1 or by its substantially identical mutants.

The native atypical p-ANCA antigen of embodiment (1) or (4) can either be tubulin beta isoform 5 or a still unknown tubulin. This has been determined by heterologous expression of the tubulin beta isoform 5 and subsequent experiments concerning the cross reactivity of the p-ANCA with this tubulin beta.

In a preferred aspect of embodiment (1), the antigen of atypical p-ANCA can be extracted by the preparation of a tubulin fraction from human or vertebrate cells. It may preferably be extracted from blood cells, cells of the immune system, from hepatocytes, cells of the bile system or from intestinal cells; especially preferred are myeloid differentiated cells, and very especially preferred are neutrophil granulocytes or cells similar to granulocytes. This tubulin preparation will especially be produced from HL-60 cells, preferably further separated by methods concerning the separation of protein mixtures, especially by the one-dimensional or the two-dimensional gel electrophoresis, very especially by the SDS gel electrophoresis and isoelectric focusing, or other methods appropriate for the purification of proteins, so that an antigen of atypical p-ANCA (atypical p-ANCA antigen) will be purified. This atypical p-ANCA antigen will be identified by immunoblotting with antisera containing atypical p-ANCA or by other appropriate methods like, for example, protein sequencing. Then, it can be used for the detection of atypical p-ANCA, for example in immunoassays or ELISAS. This tubulin preparation can be performed much easier than the nuclear envelope preparation which has been performed so far for the extraction of the atypical p-ANCA antigen.

A preferred aspect of embodiment (4) is the use of recombinantly produced TBB-5. Methods usual among experts for the recombinant production of eukaryotic proteins are used for the recombinant production of TBB-5, especially the expression as fusion protein in eukaryotic cells; very especially preferred is the expression as fusion protein with a polyhistidine tag, a c-myc tag and/or an Xpress tag. Furthermore, the use of DNA encoding for TBB-5, especially of DNA with SEQ ID NO:1, is also preferred.

Different methods for the heterologous expression of eukaryotic DNA known in the literature can be used to clone the myeloid cell-specific cDNA of TBB-5 or the native atypical p-ANCA antigen according to embodiment (7) and to recombinantly produce the protein in accordance with embodiment (10). In the present invention, mRNA has been isolated from HL-60 cells, reversely transcribed by using poly(dt) primers, amplified with the help of oligonucleotide primer specific for TBB-5 (SEQ ID NO: 14, SEQ ID NO: 15, SEQ ID NO:32, SEQ ID NO:33) for the C-terminus and the N-terminus of the TBB-5 nucleotide sequence (primer design on the basis of the database entry for TBB-5 from human brain, BC007605) and subsequently subcloned in an expression vector (e.g. PQE-Tris System [Qiagen, Hilden] or pcDNA3.1/His⁺/lacZ [Invitrogen, Karlsruhe] with integrated 6× histidine tag and/or Xpress tag [-Asp-Leu-Tyr-Asp-Asp-Asp-Asp-Lys] and/or c-myc tag [-Glu-Gln-Lys-Leu-Ile-Ser-Glu-Glu-Asp-Leu] (see also example no. 9). The result of the sequencing procedure of the cDNA corresponded to the database entry BC007605 for human TBB-5 (cerebral tissue), SEQ ID NO:1. Thus, no difference between the cDNA for TBB-5 specific for HL-60 cells and the nucleotide sequence of human brain could be detected with this method. On the one hand, this may be due to the fact that, based on today's state of knowledge, only primers could be used for the reverse synthesis of TBB-5 cDNA which had been designed on the basis of a known database sequence. A genomic analysis of the nucleotide sequence of TBB-5 specific for HL-60 cells has not yet been performed. On the other hand, an identity in the nucleotide sequence of TBB-5 independent from the cell species may be a sign of critical posttranslational modifications which are characteristic for the respective cell-specific TBB-5.

In experiments to express TBB-5 in E. coli (strain: M15; Qiagen), neither an expressed fusion protein in gels stained with Coomassie nor a reactive protein could be determined in the immunodetection with the sera containing atypical p-ANCA or with antibodies which are directed against the polyhistidine tag or the Xpress tag. On the other hand, control proteins could successfully be expressed in E. coli (e.g. FtsZ). Alternatively, a transient transfection of eukaryotic cells (e.g. HeLa cells, Cos 7 cells) has been tested. Different transfection systems like, for example, calcium precipitation and lipofection, have been contrastingly used. A significantly

better transfection efficiency could be determined when using the lipofection technique. However, an expression of full-length TBB-5 was also quantitatively restricted and inconstantly possible in the eukaryotic cells, whilst TBB-5 fragments could verifiably be expressed. Different authors describe a toxic effect of overexpressed tubulin (e.g. TBB-2 and TBB-3) in transfected yeast cells and/or a “tight” regulation of tubulin by degrading overexpressed tubulin by a rapid proteolytic decomposition ((Cleveland, D. W., Curr. Opin. Cell Biol. 1:10-14 (1989); Burke, D. et al., Mol. Cell Biol. 9:1049-1059 (1989)). The authors interpret this as a physiological reaction to prevent a quantitative disequilibrium of tubulin alpha and tubulin beta during the production process of microtubules and possibly resulting functional disorders of the cells. Due to these observations, it was assumed that full-length TBB-5 cannot be overexpressed under all conventional conditions and that at least a restricted overexpression was possible under certain expression conditions. This was confirmed in the experiment. Respective protein-chemical detection methods were used in the scope of the present invention directly after the protein expression to minimize possible protein degradation. Under these conditions, the expression and the detection of full-length TBB-5 has been successful (FIG. 6).

Based on the assumption that an elongation (e.g. by a fusion with GFP [green fluorescent protein]) or truncation of the TBB-5 nucleotide sequence could prevent the presumed posttranscriptional inactivation of the protein, transformations have been performed with truncated TBB-5 cDNA (see also example no. 10) as well as with GFP TBB-5 full-length cDNA. The expression of truncated TBB-5 cDNA and GFP TBB-5 full-length can successfully be performed (shown in FIG. 6). The immunodetection of the recombinant, truncated TBB-5 proteins by atypical p-ANCA showed that at least some (BB, BP) of these truncated TBB-5 can be detected by atypical p-ANCA (FIG. 7). The immune reactivity of BE which predominantly includes the N-terminus of TBB-5 is now the object of further experiments. GFP TBB-5 can also be detected by atypical p-ANCA (FIG. 7).

According to embodiment (4), the object of the present invention are also truncated TBB-5 whose amino acid sequence

is truncated at the N-terminus or the C-terminus by 1-220 amino acids. Preferred is a truncation by 1-100 amino acids, especially preferred are 1-50 amino acids. This applies especially to TBB-5 whose amino acid sequence is truncated at the C-terminus and/or a protein with SEQ ID NO:5, SEQ ID NO:7 or SEQ ID NO:9. Object of the invention are also the nucleic acids encoding for such molecules, preferably DNA; especially preferred is the cDNA with SEQ ID NO:4, SEQ ID NO:6, SEQ ID NO:8 as well as their substantially identical mutants.

Thus, the objects of the present invention are fusion proteins as well (according to embodiment (4)) which either contain full-length TBB-5 or truncated TBB-5. Preferred are fusion proteins with marker proteins as described above; especially preferred are fusion proteins with GFP and/or an Xpress tag, a c-myc tag or a His tag. Very especially preferred are those GFP fusion proteins containing the GFP at the C-terminus of TBB-5, especially GFP full-length TBB-5 or SEQ ID NO:29. Object of the invention are also the nucleic acids which encode for such molecules: Preferred is DNA, especially preferred is the cDNA for GFP full-length TBB-5 or SEQ ID NO:28 as well as their substantially identical mutants.

Another preferred aspect of embodiment (4) is the recombinant production of truncated TBB-5 as fusion protein with a marker protein or a peptide which can be used to isolate the fusion protein. The use of the above mentioned truncated TBB-5 is preferred. Methods which are common practice among experts for the recombinant production of eukaryotic proteins are used for the recombinant production, especially the expression as fusion protein in eukaryotic cells; very especially preferred is the expression as fusion protein with GFP, a polyhistidine tag, a c-myc tag and/or an Xpress tag. Also the use of such DNA is preferred which encodes for such truncated TBB-5 fusion proteins and whose TBB-5 protein part will be encoded by the DNA of SEQ ID NO:4, SEQ ID NO:6 or SEQ ID NO:8.

A preferred embodiment of (1) to (5) is the use of recombinantly produced atypical p-ANCA antigen based on the nucleic acid sequence SEQ ID NO:1, including its substantially identical mutants or truncated or elongated SEQ ID NO:1. The antigen of atypical p-ANCA according to the invention can thus be recombinantly produced; this way, a complex preparation of the nuclear envelope is not required for the extraction of the antigen.

This recombinant production is accomplished by common methods known among experts for the recombinant production of eukaryotic proteins, preferably by the expression as fusion protein with a peptide which serves for the isolation of the fusion protein; very especially preferred is the expression as fusion protein with a polyhistidine tag, a c-myc tag and/or an Xpress tag.

Another preferred embodiment of (1) to (5) is the use of bacterial FtsZ (SEQ ID NO:17) or of modifications of bacterial FtsZ as an antigen; bacterial FtsZ or its modifications can be obtained by the substitution, the deletion or the addition of amino acids in SEQ ID NO: 17. Preferred is the use of modifications of the bacterial FtsZ which include the parts which are highly homologous to TBB-5 on a structural level, i.e. the amino acid sequence of positions 65-135 of SEQ ID NO: 17 (for tubulin beta, corresponds to the amino acids, 95-175, homology 85-87%) and/or the amino acid sequence of positions 255-300 (for tubulin beta, corresponds to the amino acids 305-330, homology 51-78%).

Furthermore, the use of proteins or peptide fragments of FtsZ (“FtsZ peptide”) which contain conserved parts of FtsZ in several bacterial strains is also preferred. They are especially preferred if they contain the amino acid sequences of SEQ ID NO:36. Very especially preferred is the use of the peptide with SEQ ID NO:36 itself.

Especially preferred are also truncated FtsZ whose amino acid sequence is truncated by 1-180 amino acids, preferably by 1-100 amino acids; especially preferred is a truncation by 1-70 amino acids, and very especially preferred is a truncation by 1-40 amino acids at the N-terminus or the C-terminus. Furthermore preferred are FtsZ mutants in which the centre section of SEQ ID NO:17, namely the amino acid sequence of positions 136-254, has been modified by the substitution, the addition or the deletion of amino acids; 1-40 amino acids, or preferably 1-20 amino acids can be added or deleted. Object of the invention are also those nucleic acids encoding for such molecules. Preferred is DNA, especially preferred is the DNA with SEQ ID NO: 16 as well as its substantially identical mutants.

After the recombinant synthesis of FtsZ in E. coli (strain M15), a reactivity of FtsZ with sera of patients with AIH containing atypical p-ANCA could be detected in immunoblot confirmation experiments (FIG. 9, example no. 15). This reactivity could not be detected in sera of healthy control groups, patients with alcoholic cirrhosis, or other

intestinal diseases except chronic inflammatory intestinal diseases.

In addition, sera of IL-10 “knock out” mice were tested in a collective experiment which presented an animal model for ulcerative colitis (UC). Those IL-10 “knock out” mice had either been kept under sterile conditions (SPF=specific pathogen free conditions) or under standard conditions. Here, neither a reactivity against the FtsZ protein nor a reactivity with the proteins from the tubulin preparations of HL-60 cells could be detected during the immunodetection for the sera of the “sterilely” kept IL-10 mice. However, 90% of the sera of IL-10 mice with normal bacterial colonization of the intestinal area showed a reaction with the FtsZ protein as well as with the 50 kD protein in the human tubulin preparation.

Hence, the embodiments (1), (4), (5), and (11) of the invention can also be performed with FtsZ of SEQ ID NO:17 or with modified FtsZ, as described above, as the antigen of atypical p-ANCA, especially with recombinant FtsZ; Very especially preferred is the performance with FtsZ produced with the help of E. coli, mainly of E. coli M15 (Qiagen) and/or using DNA with SEQ ID NO:16.

A preferred aspect of the use of FtsZ according to the invention is the recombinant production of FtsZ. For the recombinant production of FtsZ according to embodiment (10), methods which are common practice among experts will be used for the recombinant production of bacterial proteins, especially for the expression as fusion protein after the protein induction by isopropyl-beta-D-thiogalactopyranoside (IPTG); very especially preferred is the expression as fusion protein with polyhistidine tag and the isolation by binding the polyhistidine tag to nickel exchange resins. Furthermore, the use of DNA encoding for FtsZ, especially of DNA with SEQ ID NO:16, is also preferred.

Another aspect of embodiments (1), (4), (5) and (11) of the invention is the possibility (which has been proved by the invention for the first time) not only to use the native antigen to atypical p-ANCA, which is difficult to be isolated from nuclear envelope preparations, but also to use tubulin beta isoform 5 or bacterial FtsZ or its homologues obtained by the deletion, the substitution or the addition of amino acid for the detection of atypical p-ANCA. TBB-5 or FtsZ is preferably used for this procedure. Especially

preferred is the recombinant production of the used tubulin beta 5. However, tubulin beta 5 can also be obtained from a tubulin preparation, or the used FtsZ will be recombinantly produced. DNA molecules with the sequences encoding for TBB-5 and FtsZ will be preferably used for the recombinant production, especially those with SEQ ID NO:1 or SEQ ID NO:16.

Embodiment (6) includes all proteins defined in (1) to (4) and its mutants presented above, especially all non-native proteins and fusion proteins; especially preferred are the truncated mutants of FtsZ and TBB-5 as well as their fusion proteins. Very especially preferred are proteins with amino acid sequences of SEQ ID NO:5, SEQ ID NO:7, SEQ ID NO:9 and SEQ ID NO:29. Embodiment (6) does specifically not include the already described proteins TBB-5 and FtsZ, especially not TBB-5 with the amino acid sequence of SEQ ID NO:2 or FtsZ with the amino acid sequence of SEQ ID NO:17.

Embodiment (7) includes the DNA encoding for embodiment (6), especially the non-native DNA sequences.

All vectors known in the literature which are used for the heterologous amplification or expression of DNA are appropriate for the execution of the invention according to embodiment (8). Vectors for the expression of eukaryotic proteins are especially preferred for the use of cDNA strands encoding for TBB-5 or the native antigen of atypical p-ANCA or for their derivates according to the invention. Very especially preferred are vectors which provide the heterologously expressed protein with a polyhistidine tag, a c-myc tag or an Xpress tag. On the other hand, the vectors according to (8) which include the DNA of FtsZ and its derivates according to the invention are preferred for the use in known prokaryotic expression systems and especially allow the induction of the expression by isopropyl-beta-D-thiogalactopyranoside (IPTG) and/or the expression of FtsZ as fusion protein with polyhistidine tag.

In the case of FtsZ and its derivates according to the invention, the host organisms according to embodiment (9) are preferably prokaryotes. In the case of TBB-5 and its derivates according to the invention, the host organisms for the amplification of the DNA or the vectors are preferably prokaryotes; eukaryotes or yeasts will preferably be used for the expression. Very especially preferred are prokaryotic E. coli strains

and eukaryotic mammalian cells, in the latter case especially HeLa cells and Cos 7 cells.

The production according to embodiment (10) is accomplished in compliance with the protocols appropriate for the respective expression systems, in the case of eukaryotic cells either by permanent or transient transfection, preferably by transient transfection. The expression products will then be separated from the host cells and further purified, preferably by the gel electrophoresis and/or by affinity chromatography.

Being one aspect of the embodiments (1) to (5) and (11), the atypical p-ANCA antigen according to the invention for the production of highly specific, highly sensitive and reproducible assays, especially solid phase assays, can be used for the detection of atypical p-ANCA. For the first time, the identification and characterization of this antigen, especially of its sequential homologies with tubulin beta isoform 5, allows the development of such solid phase assays specific for atypical p-ANCA which presents a significant improvement and especially a significant simplification in comparison with the so far usual diagnostic methods. Preferably, those assays are based on the principle of the ELISA technology.

The detection method for atypical p-ANCA according to embodiment (1)-(5) is a method in which a sample comes into contact with the antigen to atypical p-ANCA so that atypical p-ANCA contained in the sample are bound to the antigen to atypical p-ANCA. The bound atypical p-ANCA or the bound atypical p-ANCA antigen will be detected with appropriate methods. These methods will preferably be performed in vitro.

Appropriate methods for the execution of the detection of bound atypical p-ANCA or bound atypical p-ANCA antigens are preferably immunological methods (according to embodiment (5)). The quantitative determination of the concentration of atypical p-ANCA in a sample according to the invention can especially be accomplished with the help of an immunoassay; especially preferred is the use of an ELISA, and/or the determination procedure includes the comparison with a standard concentration of atypical p-ANCA.

Thus, such method includes, for example, in the first step the production of the sample to be tested, for example by withdrawing a

body fluid, especially blood, and its treatment, especially the extraction of serum. In the second step, the sample will be brought into contact with the atypical p-ANCA antigen which is preferably bound on a solid phase. After the incubation, the supernatant will be removed. The amount of atypical p-ANCA bound to the substrate over the antigen can then be determined by reproducible and known immunoassay methods like, for example, ELISA.

In a preferred embodiment of (1) to (5), a specific secondary agent will be used which detects and binds an epitope of the atypical p-ANCA without affecting its ability to bind the atypical p-ANCA antigen. Secondary agents preferably include antibodies (secondary antibodies). Especially monoclonal or polyclonal antibodies can be used as secondary antibodies. Especially preferred are polyclonal antibodies. Secondary antibodies are human or murine antibodies, antibodies from rats, rabbits, sheep, goats or pigs, but preferably goat or sheep antibodies. The secondary agents can be coupled to a detector reagent which allows the detection of the agent as well as the quantitative and qualitative evaluation of the bond of the secondary agent, especially selected from enzymes, fluorescence dyes and radio isotopes; especially preferred are horseradish peroxidase or fluorescein. Parts of the immunobinding couple (e.g. of biotin/streptavidin etc.), magnetic beads, and other functional connections are eligible as functional connections which allow the detection of the bond of a secondary agent to atypical p-ANCA and/or serve for the isolation of the secondary agent or a conjugate of a secondary agent and atypical p-ANCA. Especially preferred as a secondary agent is a polyclonal Goat Anti Human IgG conjugated with fluorescein isothiocyanate (FITC) or a horseradish peroxidase-coupled Sheep Anti Human IgG, especially the respective IgG(H+L).

In another preferred embodiment of (1) to (5), the atypical p-ANCA antigen is coupled to a carrier material by appropriate immobilization methods. Such appropriate immobilization methods include adequate coupling techniques which do not modify the specificity of the antigen, like, for example, the covalent cross-linking of the antigen with the carrier material. An alternatively preferred embodiment is the

immobilization of the antigen to a carrier material by interacting with the secondary antibody according to the invention.

The diagnostic methods according to embodiment (12) can be performed in vivo and in vitro, but the in vitro method will be preferred. For the use according to embodiment (12), an atypical p-ANCA antigen according to embodiment (6) will be preferably used, especially native atypical p-ANCA antigen, tubulin beta isoform 5 or FtsZ as well as their truncated mutants. The atypical p-ANCA antigen can be purified from sources in which it naturally exists, or it can be recombinantly produced. Recombinant antigen will preferably be used.

In addition to the protein defined in embodiment (6), the kit according to embodiment (11) may contain a microbial strain of the cell line for the production of this protein. Furthermore, this kit preferably contains bacterial FtsZ, TBB-5, elongated or truncated TBB-5 and/or a microbial strain of cells which can be used for the recombinant production of FtsZ, TBB-5, or elongated or truncated TBB-5. In addition to these components, the kit may also include a secondary agent as defined above, preferably a secondary antibody as defined above, agents for the detection of the bond of atypical p-ANCA to the secondary agent over the antigen, buffers and/or culture media.

A preferred use of the atypical p-ANCA antigen according to embodiment (12) is its use for the detection of chronic inflammatory intestinal diseases, especially ulcerative colitis (UC) and/or Crohn's disease, and/or autoimmune liver diseases, especially autoimmune hepatitis (AIH) and/or primary sclerosing cholangitis, especially for the detection of AIH and primary sclerosing cholangitis (PSC). It could surprisingly be detected that atypical p-ANCA are the only relevant seromarkers for PSC in the entire diagnostic armentarium. Atypical p-ANCA could be detected with high prevalence in the sera of patients with PSC (94%). Except for one tested serum, all sera containing p-ANCA (97%) showed a fluorescence pattern of atypical p-ANCA with median serum titers of 1:320. Only atypical p-ANCA proved to be relevant seromarkers for the diagnosis of PSC, compared to other antibodies like, for example, antinuclear antibodies (ANA) and smooth muscle antibodies (SMA) (table 3 and 4, example no. 16).

The atypical p-ANCA antigen according to embodiment (12) is also preferably used in the diagnostic procedure of autoimmune hepatitis (A1H). Atypical p-ANCA have been identified as seromarkers with the highest diagnostic precision for the diagnosis of AIH (table 3 and 4, example no. 16).

The invention will be further explained on the basis of the following examples. However, these examples do not restrict the scope of the invention.

### EXAMPLES

### Example 1

### Patient Sera for the Identification of the Target Antigen of Atypical p-ANCA

In this study, serum samples of 37 patients with autoimmune liver diseases (primary sclerosing cholangitis [PSC] n=7, autoimmune hepatitis [AIH] n=30) and sera of 5 healthy control persons have been used. Until being used, all sera were stored at a temperature of −20° C.

The diagnoses of the patients were based on established, clinical, endoscopic, histological, radiological, and serological criteria (Angulo, P. et al., J. Hepatol. 32:182-187 (2000); Wiesner, R. H., in: Krawitt, E. L et al., Hrsg., Autoimmune Liver Diseases, 2nd Ed., Amsterdam, Elsevier, 381-412 (1998)). The activity level of the disease for PSC and AIH was determined by established measuring values based on clinical data and laboratory data like the “Mayo Risk Score” for primary sclerosing cholangitis (Kim, W. R. et al., Mayo Clin. Proc. 75:688-694 (2000)) and the “Scoring System for the Diagnosis of Autoimmune Hepatitis”, suggested by the “International Autoimmune Hepatitis Study Group” (Alvarez, F. et al., J. Hepatol. 31:929-938 (1999)). The clinical and biochemical characteristics of the participants in the study at the time of the withdrawal of the serum sample are summarized in table 1. All data will be specified either as mean value±standard error of the mean value or as median.

All steps of the study have been performed in accordance with the valid version of the Helsinki declaration of 1975. The study has been approved by the Ethics Committee of the University of Bonn (Germany) responsible for the examination.

TABLE 1
Characteristics of the study participants

| | | | Healthy |
|---|---|---|---|
| | | | control |
| | PSC | AIH | persons |
| Number (total n = 42) | 7 | 30 | 5 |
| Sex (f/m) | 2/5 | 19/11 | 3/2 |
| Average age in years (range) | 51 (37-56) | 44 (20-73) | 32 (26-42) |
| Associated diseases | | | |
| Concomitant chronic inflammatory | 4 | 1 | — |
| intestinal disease | | | |
| Concomitant PSC | — | 2 | — |
| Occurrence of autoantibodies | | | |
| ANCA positive | 7 | 30 | 0 |
| Classic p-ANCA | 0 | 3 | 0 |
| Atypical p-ANCA | 7 | 27 | 0 |
| Median titer of the atypical | 640 | 640 | — |
| p-ANCA (range) (≧1:20) | | | |
| ANA positive (≧1:40) | 4 | 20 | — |
| SMA positive (≧1:80) | 5 | 16 | — |
| Biochemical characteristics | | | |
| (x ± SEM) | | | |
| Bilirubin (−1.0 mg/dl) | 3.7 ± 1.9 | 2.2 ± 2.1 | 0.8 ± 0.3 |
| GPT (5-34 U/1) | 41 ± 11 | 47 ± 16 | 23 ± 6 |
| Alkaline phosphatase (40-190 U/1) | 515 ± 317 | 166 ± 144 | 116 ± 13 |
| Mayo Risk Score* | 3.69 ± 0.59 | — | — |
| Disease activity | | | |
| Active/inactive | 7 | 19/11 | — |
| Therapy at the time of the | | | |
| examination | | | |
| No therapy | 0 | 1 | 0 |
| Immunosuppressive drugs | 0 | 29 | 0 |
| Ursodeoxycholic acid | 7 | 3 | 0 |

*The Mayo Risk Score indicates the risk to die: <0 or =0: low risk; 0-2: medium risk; >2: high risk

### Example 2

### Culture of Promyelomonocytic HL-60 Cells and Other Cell Cultures

Human promyelomonocytic HL-60 cells (Deutsche Sammlung von Mikroorganismen und Zellkulturen GmbH (DSMZ) [German Resource Center for Biological Material], Braunschweig; ACC-3) were cultivated in RPMI medium (PAA, Pasching, Austria) with 10% (v/v) heat inactivated fetal calf serum at 5% of CO₂ and a temperature of 37° C. (Collins, S. J. et al., Nature 270:347-349 (1977)). Since more than 50% of the cells spontaneously differentiated into mature granulocytes, a chemical induction of the differentiation, for example with 1% (v/v) dimethylsulfoxide, over 4-6 days was renounced.

The following myeloid differentiated cell lines can be used as an alternative to HL-60 cells: Murine 32 D cells, U937, K-562. Some experience with murine 32 D cells have been made in the scope of the first protein-chemical experiments (Terjung, B. et al., Gastroenterology 119:310-322 (2000)). Due to the limited availability and the comparatively large number of required cells, neutrophil granulocytes from human blood are only partially appropriate as test medium.

Human HeLa cells (DSMZ; ACC-57) have been cultivated in 90% RMPI 1640 medium with 10% (v/v) heat inactivated fetal calf serum, and simian Cos-7 cells (DSMZ; ACC-60) have been kept in 90% RMPI 1640 with 10% (v/v) heat inactivated fetal calf serum at 5% of CO₂ and at a temperature of 37° C. (Scherer, W. F. et al., J. Exp. Med. 97:695-710 (1953); Gluzman, Y., Cell 23:175-182 (1981)). Every 3-5 days, the cells were passaged. They were mixed with a trypsin/EDTA solution (5 U of trypsin/ml) to be able to dissociate the adherent cells from the bottom of the cell culture bottle.

Human hepatoblastoma liver cells (Hep G2; ATCC HB-8065) were cultivated in RPMI medium which was supplemented with 10% of heat inactivated fetal calf serum and grown until the stadium of confluence. Every 3-5 days, the cells were passaged by trypsin digestion.

### Example 3

### Production of Nuclear Envelope Extracts from HL-60 Cells

The production of the nuclear envelope extracts followed the modified standard protocols (Gerace, L. et al., J Cell Biol 95:826-837 (1982); U.S. Pat. No. 6,627,458; Dwyer, N. et al., J. Cell Biol. 70:581-591 (1976); Gerace, L et al., J. Cell Biol. 95:826-837 (1982)). Here, the harvested cells were incubated in hypotonic lysing buffer (10 mM Tris HCl, pH value 7.5; 1 mM of MgCl₂; 1 mM of dithiothreitol) which has been mixed with proteinase inhibitors (1 μg/ml of aprotinin, bacitracin, benzamidin, leupeptin, pepstatin). Subsequently, the cell membranes and the nuclear membranes have been disrupted with a Pestle Homogenisator (Fisher Scientific, Pittsburgh, USA). To separate the nuclei from the cytoplasmic parts of the HL-60 cells, the cell suspension has been underlayered with 30% (w/v) sucrose and ultracentrifugated (35,000 rpm, 60 min.). The pellet containing the nuclei was incubated in a nuclear extraction buffer (20 mM of Tris HCl, pH value 7.5; 0.5 mM of MgCl₂; 1 mM of dithiothreitol; protease inhibitors as described above), and the DNA and RNA attached to the nuclear envelope was digested for 15 minutes with DNAse (1 μg/ml) and RNAse (10 μg/ml) at room temperature (RT). The nuclear envelope components could then be separated from the DNA-associated nuclear parts during the following centrifugation (13,000 rpm, 20 s). For the further purification, the nuclear envelope extracts were washed with a nuclear extraction buffer with and without 0.5 M of common salt, and parts of the nuclear membrane were sonicated for 20 seconds with a Bandelin sonicator (Bandelin MS73 Sonicator, Bandelin Electronic, Berlin, Germany). The nuclear envelope fraction was diluted at a ratio of 1:1 with sample buffers (250 mM of Tris HCl, pH value 6.8, 4% (w/v) SDS, 0.005% (w/v) bromophenol blue, 20% (v/v) glycerol, 5% (v/v) β mercaptoethanol). The extracts were stored at a temperature of −20° C.

### Example 4

### Production of Tubulin Extracts from HL-60 Cells, HeLa Cells and COS-7 Cells

So far, only standard protocols are available for the extraction of tubulin from bovine brain (Williams, R. C. und Lee, J. C., Meth Enzymol 85:376-385 (1982); Detrich, H. W. et al., J. Biol. Chem. 260:9479-9490 (1985)). These protocols were used in slightly modified form for the extraction of tubulin from HL-60 cells, HeLa cells and COS-7 cells. The pellet obtained after the centrifugation of the cells (3,000 rpm, 4° C.) was washed with a phosphate buffer and then inserted into the so-called “microtubule buffer” (0.1 M of 2 (N-morpholino)-ethanesulfonic acid; 1 mM of EDTA, 0.5 mM of MgCl₂, pH value 6.5), solubilized with a “Dounce” homogenizer and then centrifugated (13,000 rpm, 1 hour, 4° C.). The same amount of cold microtubule buffer as well as 8 M of glycerol solution was added to the supernatant. After the addition of 1 mM of GTP which causes a polymerization of the tubulin, the solution was incubated for 30 minutes at a temperature of 37° C.; then, the solution was centrifugated (24,000 rpm, 25° C.). The obtained pellet, consisting of polymerized tubulin, was then mixed with microtubule buffers or, depending on its further intended use, sample buffers and stored at a temperature of −20° C. until its further use, if required.

### Example 5

### Reducing One-Dimensional SDS PAGE Gel Electrophoresis with Western Blotting and Subsequent Chemiluminescence

The obtained cell extracts (nuclear envelope extracts as well as tubulin preparations) were separated under reducing conditions using the SDS polyacrylamide gel electrophoresis (PAGE). This procedure followed the standard protocol by Laemmli (Laemmli, U. K., Nature 227:680-685 (1970)) (stacking gel 4%, resolving gel 10%, 150 V). Approx. 10-15 μg of the used protein were applied per line. A commercial marker was used as molecular weight marker for the range 10-250 kD (BIORAD, Hercules, USA, Cat. no. 161-0372). The electrophoretically separated proteins were detected with a Coomassie gel stain (0.05% (w/v) Coomassie Brilliant Blue R, 50% (v/v) ethanol, 10% (v/v) acetic acid) or with silver nitrate stain (Silver stain plus, BioRad, Hercules, USA).

After having finished the electrophoresis, the electrophoretically separated proteins were transferred to a nitrocellulose membrane with the help of a semi-dry blot apparatus during the performed Western Blotting procedure. The standard procedure by Towbin was used to select the required buffers (Towbin, H. et al., Proc. Natl. Acad. Sei. USA 76:4350-4354 (1979)). Reactive proteins were detected by means of the immunodetection and a subsequent chemiluminescence reaction: Incubation for 45 minutes at room temperature with primary antibody, e.g. atypical p-ANCA, diluted at a ratio of 1:200-1:1,500 in blocking solution (5% (w/v) non-fat dry milk in PBS, 0.01% (v/v) Tween®-20), then washing with PBS, 0.01% (v/v) Tween®, then detection with a secondary antibody, e.g. horseradish peroxidase-coupled Sheep Anti Human IgG(H+L) secondary antibody (diluted at a ratio of 1:5,000) and oxidation of luminol in the presence of hydrogen peroxide (ECL Western Blotting detection reagents, Amersham Pharmacia Biotech). The reactive proteins of the nitrocellulose membrane were made visible on a film especially developed for the chemiluminescence reaction.

### Example 6

### Two-Dimensional Gel Electrophoresis

During the first dimension of the two-dimensional gel electrophoresis, the so-called isoelectric focusing, the proteins were separated according to their isoelectric point. In the second dimension, an SDS PAGE electrophoresis (see also example no. 5), the protein mixture was additionally separated according to the molecular weight. Commercial polyacrylamide strips with an immobilized pH gradient (e.g. pH value 4-7, pH value 4.7-5.9, length 11 cm; Ready Strips IPG, Biorad) were used for the isoelectric focusing. The gel strips were incubated for 12 hours with a patient sample (sample diluted at a ratio of 1:1 with a sample buffer (8 M of urea, 2% (w/v) CHAPS, 50 mM of dithiothreitol, 0.2% Bio-Lyte-Ampholyte)). In the following, the proteins were separated according to their pI value at 20,000 Vh (volt hours). A commercial standard was used as IEP standard for the determination of the IEP in the range of pI value 4.45-9.6 (BIORAD, Hercules, USA, Cat. no. 161-0310). In the second dimension, an SDS PAGE electrophoresis was performed (4% stacking gel, 10% resolving gel, 200 V, 45 min.).

Before the gel strips were applied to the stacking gel, the gels were equilibrated for 10 minutes at room temperature in two different buffers (buffer no. 1: 6 M of urea, 0.375 M of Tris HCl, pH value 8.8, 2% (w/v) SDS, 20% (v/v) glycerine, 2% (w/v) DTT; buffer no. 2: 6 M of urea, 0.375 M of Tris HCl, pH value 8.8, 2% (w/v) SDS, 20% (v/v) glycerine, 2.5% (w/v) iodoacetamide). The isoelectric focusing was performed in accordance with standard protocols (O'Farrel, P. H., J. Cell Biol. 250:4007-4021 (1975); Gorg, A. et al., Electrophoresis 20:712-717 (1999)).

Alternatively, urea/carrier ampholyte gels (9.2 M of urea, 4% acrylamide, 20% Triton® X, 1.6% ampholytes pH value 5-7, 0.4% ampholytes pH value 3-10, 0.01% ammonium persulphate, 0.1% TEMED) produced in glass capillaries were used in previous test (U.S. Pat. No. 6,627,458).

The proteins were detected by staining the gels and/or immunoblotting and chemiluminescence reactions as described in example no. 5.

When separating the nuclear envelope preparations from example no. 3, two “spots” with an approximate molecular weight of 50 kD and an isoelectric point between 4.9 and 5.1 were identified on a regular basis (FIG. 1). Those spots were also detected by the majority (>94%) of the tested sera of patients with PSC or AIH (example no. 1) containing atypical p-ANCA (32 of 34 tested sera with atypical p-ANCA, thereof AIH: 26/27 and PSC 6/7). None of the sera of ANCA-negative healthy individuals (n=5) reacted with these 50 kD proteins. Nor did sera of patients with AIH or PSC and classic p-ANCA (n=3) detect the 50 kD protein.

### Example 7

### Affinity-Purified Atypical p-ANCA

To produce affinity-purified atypical p-ANCA, tubulin (see example no. 4) or nuclear envelope preparations (see example no. 3) were preparatively separated with the help of a one-dimensional SDS PAGE (see example no. 5). Subsequently, the proteins were transferred to a nitrocellulose membrane by semi-dry Western Blotting, and a reactive band was immunodetected by the incubation with a serum containing atypical p-ANCA (Olmsted, J. B., J. Biol. Chem. 256:11955-11957 (1981)). This reactive band was cut out from the nitrocellulose membrane. Bound atypical p-ANCA were eluted for 20 seconds with an acid solution from 200 mM of glycine (pH value 2.8) and 1 mM of EDTA. The obtained solution was then neutralized with 1 M of TrisBase to pH value 7.4. For the immunofluorescence microscopy, the affinity-purified antibodies were used undilutedly. The staining pattern obtained after the indirect immunofluorescence microscopy (see also example no. 13) was compared with the staining pattern of atypical p-ANCA which were not affinity-purified.

### Example 8

### Preabsorption of Atypical p-ANCA with Tubulin Extracts from HL-60 Cells

Serum containing atypical p-ANCA, classic p-ANCA or no ANCA was preabsorbed with a tubulin preparation (see also example no. 4) at a ratio of 1:10 for 30 minutes at a temperature of 37° C. After the centrifugation (13,000 rpm, 30 min.), the supernatant was used for immunofluorescence experiments. The obtained fluorescence patterns were analyzed with the indirect immunofluorescence microscopy (see also example no. 13.).

### Example 9

### Cloning the cDNA of Tubulin Beta 5 Specific for HL-60 Cells and Expression as Recombinant Protein

The respective cDNA for cloning the myeloid cell-specific tubulin beta isotype 5 was produced with the help of an oligonucleotide primer for tubulin beta 5 after the extraction of RNA from human HL-60 cells. The different steps were as follows:

A) Extraction of total RNA from HL-60 cells: The total RNA from HL-60 cells was isolated according to standard procedures (e.g. RNeasy Kit, Qiagen, Hilden).

B) Isolation of polyadenylated mRNA: The polyadenosine nucleotide sequence which is located at the 3′ end of most mRNAs was bound covalently to cellulose fibers over complementary oligo(dT)nucleotides and could

thus be separated from the ribosomal RNA and the total RNA (e.g. Omniscript Kit, Qiagen).

C) cDNA synthesis with the help of reverse transcription: Single-strand cDNA was produced from the polyadenosine mRNA by reverse transcription using reverse transcriptase and oligo(dT) random primers.

D) Amplification of the single-strand cDNA with the help of PCR and oligonucleotide primers against the tubulin beta 5 genetic sequence: A PCR (Polymerase Chain Reaction) was performed against sections at the N-terminus and the C-terminus of tubulin beta 5 (primer sequences with SEQ ID NO:14 and SEQ ID NO:15) over 35 cycles in a thermal cycler with Taq polymerase and oligonucleotide primers according to standard protocols. The amino acid sequence or nucleotide sequence of tubulin beta 5 (NCBI BC007605, human brain) available from the database was used in this process. The PCR amplification product was separated in an agarose gel in comparison with a length standard. Then, the cDNA was eluted from the gel, purified from contaminating PCR products with the “Quiaquick Gel Extraction Kit” (Qiagen), and finally cloned into a prokaryotic/eukaryotic vector (e.g. PQETriSystem [Qiagen, Hilden] or pcDNA3.1/His⁺/lacZ [Invitrogen, Karlsruhe]) with an integrated 6× histidine tag, an Xpress [-Asp-Leu-Tyr-Asp-Asp-Asp-Asp-Lys] tag and/or a c-myc tag [-Glu-Gln-Lys-Leu-Ile-Ser-Glu-Glu-Asp-Leu-]). In the following, competent E. coli were transformed with the donor plasmid. Respectively, eukaryotic cells were transiently transfected with the donor plasmid.

E) Expression of the recombinant protein in eukaryotic systems: Since posttranslational modifications were assumed due to previous examination results, only eukaryotic expression systems (e.g. HeLa cells, COS-7 cells) were used after the missing protein expression in the prokaryotic system. The transient transfection of adherent eukaryotic cells, e.g. HeLa cells or COS7 cells, was performed by lipofection according to optimized standard protocols (Rose, J. K. et al., Bio Techniques 10:520-525 (1991)). 3×10⁵ HeLa cells/COS7 cells were plated in a 6-well plate in 2 ml of RPMI 1640 medium. These cells were then incubated (37° C., 5% CO₂) until the cells were confluent at a percentage of 70-90% (24 hours). The cDNA plasmid to be transfected (3 μg in 200 μl of OPTIMEM medium, Invitrogen) was incubated at room temperature for 20 minutes together with the transfection medium (approx. 6 μl; e.g. Transfektin Lipid Reagenz [Biorad] diluted in 200 μl of OPTIMEM medium) so that a sufficient number of complexes from DNA and transfection reagent could be

produced. Subsequently, 400 μl of this mixture were applied to each well of the 6-well plate and incubated for 48 hours, as required (37° C., 5% CO₂).

Fusion proteins which have six histidine rests or the Xpress tag at their N-terminus or the c-myc tag at the C-terminus respectively were expressed from the transfected cells. After having produced the lysates of the transfected HeLa cells or Cos 7 cells, the tubulin beta 5 fusion protein marked by polyhistidine and Xpress was separated from the other cell proteins and eluted depending on the pH value. Subsequently, the recombinant protein was separated with the help of the SDS PAGE electrophoresis (see example no. 5) and either detected with the Coomassie blue gel staining or transferred to a nitrocellulose membrane by semi-dry Western Blotting. The proteins bound to the nitrocellulose membrane were either detected with antibodies against the polyhistidine tag, antibodies against the Xpress tag, antibodies against the c-myc tag or by atypical p-ANCA from sera with the help of the immunodetection procedure and the chemiluminescence method.

### Example 10

### Cloning the cDNA of Truncated TBB-5 Fragments, GFP TBB-5 and TBB-5 c-myc

The same methods were used for the recombinant production of truncated TBB-5 fragments, of GFP TBB-5 and TBB-5 c-myc (full-length TBB-5 with c-myc tag) as well as for the cDNA synthesis of full-length TBB-5 (example no. 9 A-D). The cDNA of TBB-5 c-myc (SEQ ID NO:34), GFP TBB-5 (SEQ ID NO:28) as well as the cDNA of three fragments of TBB-5 showing different lengths were synthesized (see also FIG. 6B). These fragments were defined as BP (SEQ ID NO:8), BB (SEQ ID NO:6) and BE (SEQ ID NO:4).

a) BP (includes the 3′ end of TBB-5, approximately 40% of the total nucleotide sequence): A PCR with Taq polymerase (HotStar Taq DNA polymerase [Qiagen] was performed according to standard protocols with the following linker primers (elongation 54/62° C., synthesis: 45 s):

KpnI Site:

(SEQ ID NO:12)

forward 5′-gcggtacctttctttatgcctggctttg-3′
EcoRI Site:

(SEQ ID NO:10)

backwards 5′-gcggtacctttctttatgcctggctttg-3′

b) BB (5′ end of TBB-5, approximately 70% of the total nucleotide sequence of TBB-5): First, the cDNA synthesis of full-length TBB-5 was accomplished; then, a restriction digestion was performed with the help of BamHI.

c) BE (truncated 5′ end of TBB-5, approximately 47% of the total nucleotide sequence of TBB-5): First, the cDNA synthesis of full-length TBB-5 was accomplished; subsequently, a restriction digestion was performed through EcoRI.

d) GFB TBB-5 (BG; full-length TBB-5+°green fluorescent protein [GFP]): First, the cDNA synthesis of full-length TBB-5 was accomplished; then, another PCR was performed for GFP with the following linker primers according to standard protocols (elongation 57/65° C.):

KpnI Site:

(SEQ ID NO:30)

forward 5′-gcggtaccatgagcaagggcgaggaac-3′
EcoRI Site:

(SEQ ID NO:31)

backwards 5′-gcgaattccttgtacagctcgtccatg-3′

e) TBB-5 c-myc (full-length TBB-5 with c-myc tag): First, the cDNA synthesis of full-length TBB-5 was accomplished with the following linker primers (elongation 60/72° C.):

KpnI Site (forward)

(SEQ ID:32)

5′ ggggtaccgagggaaatcgtgcacatccaggctg-S′
EcoR I Site (backwards)

(SEQ ID:33)

5′ ggaattctcacagatcttcttcagaaataagtttttgttcggcctcct
cttc ggcctcctc-3*

The obtained cDNA products were purified (QIAquick PCR Purification Kit, Qiagen). The expression vector pcDNA3.1 (Invitrogen) was cut over specific restriction enzyme interfaces (NcoI and XhoI or KpnI and EcoRI). Then, the used enzymes were inactivated for 10 min. at a temperature of 65° C., and the obtained vector fragments were purified (GeneElute Gel Purification Kit [Sigma]). The success of the restriction digestion was checked on a 2% agarose gel. The cDNA fragments of the individual TBB-5 fragments or the GFP TBB-5 were ligated into the pcDNA3.1 vector adding T4 DNAse (60 min., 22° C.), and E. coli was transformed with this vector (Rapid DNA Ligation & Transformation Kit, Fermentas). The suspension from E. coli and plasmid was incubated on an LB agar plate over night (37° C.). Grown clones were picked and cultivated over night at a temperature of 37° C. in LB medium which had been mixed with ampicillin (final concentration 100 ng/ml). Two of these over night cultures were centrifugated for 5 min. at 3,000 rpm. The obtained pellet was resuspended with 500 μl of a solution from 10 mM of Tris HCl (pH value 8.0) and 1 mM of EDTA (pH value 8.0); then, the bacteria were lysed by adding 500 μl of a solution consisting of 0.2 M of NaOH and 1% (w/v) SDS (60 min. at room temperature), they were neutralized by adding a solution (700 μl) from 3 M of potassium acetate (pH value 4.8) and finally centrifugated (10 min., 13,000 rpm) (Plasmid Preparation Mini Kit, Qiagen). Subsequently, the solution was added to 700 μl of isopropanol and centrifugated for 2-3 minutes

at 13,000 rpm; the dried pellet was added to 50 μl of H₂O/RNAse (100 μg/ml). To obtain large quantities of plasmids, the Mega Kit: NucleoBond® PC 2000 by Macherey & Nagel was used. Furthermore, the plasmid DNA of the obtained clones of BP, BB, BE and GFP TBB-5 was again checked with the help of a sequencing procedure (SeqLab, Extended Hot Shot).

Subsequently, the transfection of eukaryotic cells, e.g. simian Cos-7 cells or human HeLa cells, was accomplished by lipofection according optimized standard protocols (Rose, J. K. et al., Bio Techniques 10:520-525 (1991)) (see also example no. 9E). The fusion proteins expressed from the cells were marked by an 8× polyhistidine tag as well as by the so-called Xpress tag (-Asp-Leu-Tyr-Asp-Asp-Asp-Asp-Lys) and/or the c-myc tag (Glu-Gln-Lys-Leu-Ile-Ser-Glu-Glu-Asp-Leu). Proteins marked with polyhistidine were extracted from the cell extracts with the help of nickel exchange resins. The protein extracts were then added to sample buffers and examined with a one-dimensional and two-dimensional gel electrophoresis and, if required, a subsequent immunodetection for their reactivity with antibodies which are directed against the integrated polyhistidine tag (diluted at a ratio of 1:1,000), or with antibodies which are directed against the Xpress tag (diluted at a ratio of 1:1,000), or with ANCA-positive sera. In other fluorescence microscopic experiments, the TBB-5 c-myc was mainly detected in the nucleus of transfected cells with monoclonal antibodies which are directed against the epitope AS 408-439 at the C-terminus of the c-myc tag (c-myc 9E10: sc-40, Santa Cruz Biotechnology; diluted at a ratio of 1:500-1:1,000).

### Example 11

### Isolating the Antigen of Atypical p-ANCA and “Peptide Mass Fingerprinting”

“Peptide mass fingerprinting” (Bernardo, K. et al., Antimicrob. Agents Chemother. 48:546-555 (2004); Appel, R. D. et al., Trends Biochem. Sei. 19: 258-260 (1994)) was performed in two different laboratories (Center for Molecular Medicine, University of Cologne, Germany, and SWISS-2D Service, Central Laboratory for Clinical Chemistry, University of Geneva, Switzerland).

A) In-gel digestion: Reactive protein spots, stained with Coomassie blue (Biorad), were cut from the gel, broken up and purified with acetonitrile/water (1:1 (v/v)). The gel pieces were shrunk with pure acetonitrile, soaked in 50 mM of NH₄HCO₃ and dried in a vacuum (Speedvak).

10 mM of dithiothreitol (DTT) were added to 50 mM of NH₄HCO₃, and the proteins were reduced for 45 min. at a temperature of 56° C. For the alkylation of reduced cysteine residues, the residual liquid was removed, the same volume of 50 mM iodoacetamide was added to 50 mM of NH₄HCO₃ and incubated for 30 minutes in the dark. Before the in-gel digestion, the gel pieces were purified and dried as described above. Then, they were incubated for one hour on ice in an ice-cold solution of 12.5 ng/μl of trypsine in 25 mM of NH₄HCO₃ and 10% (v/v) acetonitrile; finally, they were incubated over night at a temperature of 37° C. The digestion was stopped by adding 5-20 μl of 1% trifluoracetic acid (TFA). The peptides were extracted with TFA for 30 minutes at a temperature of 37° C.

B) MALDI-TOF mass spectrometry of the proteins digested in gel: The extracted proteins (0.4 μl) were mixed with 1.2 μl 5 mg/ml of 2,5-dihydroxybenzoic acid in 0.1% TFA/acetonitrile (ratio 2:1) and dropped on an 800 μm target. Positive ion spectra were recorded on a Reflex IV Matrix Assisted Laser Desorption (MALDI) Time of Flight (TOF) mass spectrometer (Bruker Daltonics, Bremen, Germany) in reflectron mode. A peptide calibration standard was used for the internal calibration of a mass spectrum from m/z 1046 to m/z 3147. The evaluation software XMASS 5.1.1 was used for the optional internal recalibration of trypsine autolysis peak values and for the generation of peak value lists.

C) Database search: The identification of the proteins after the MALDI-TOF Fingerprinting procedure was accomplished either by research in the NCBInr public database (National Center for Biotechnology Information, Bethesda, Mass.), the SmartIdent database (Expert Protein Analysis System [Expasy] proteomics Server of the Swiss Institute of Bioinformatics, Basel, Switzerland) and/or the MASCOT search engine for rapid protein identification (version 1.9, Matrix Science, London, UK). Alignments of amino acid sequences were created with CLUSTAL W (version 1.81, Multiple Sequence Alignments Program of the Center for Molecular and Biomolecular Informatics, Nijmegen, The Netherlands).

### Example 12

### Comparison of the Atypical p-ANCA Antigen According to the Invention with the Antigen Fraction which has been Isolated According to U.S. Pat. No. 6,627,458

The nuclear envelope protein described in U.S. Pat. No. 6,627,458 with an approximate molecular weight of 50 kD and an approximate pI of 6.0 has not been analyzed by mass spectrometry so that it cannot be proved or disproved if the above mentioned protein (50 kD, pI 6.0) is tubulin beta isotype 5 or not. Another comparable test with HL-60

nuclear envelope extracts which have been separated with the currently used procedure for the two-dimensional gel electrophoresis also showed reactive spots in the alkaline spectrum of pH value 5.5-6.0 in addition to the characteristic reactive spots in the spectrum of pH value 4.7-5.1 which have been identified as tubulin beta isotype 5 by the present invention.

In U.S. Pat. No. 6,627,458, it was tried with the help of the two-dimensional gel electrophoresis to further separate the complex protein mixture of the nuclear envelope preparation (first dimension: isoelectric focusing with separation of the proteins according to their isoelectric point; second dimension: SDS PAGE gel electrophoresis with separation according to the molecular weight). In contrast to the subsequently used procedures, the pH gradients of the gels used during the isoelectric focusing were produced in glass capillaries. In addition to several other technical problems, the pI gradients could not be clearly reproduced, and thus, the proteins could not be reliably separated and allocated to a pI value. Thus, this procedure has been replaced in that form that immobilized pH gradients were then used on gel strips (see also example no. 6). First, the protein mixture of the nuclear envelope extracts was separated with the method mentioned first showing a large number of reactive proteins with a molecular weight of 50 kD and different pI values in the gels stained with silver nitrate. A carrier ampholyte mixture was used for the gel production for the isoelectric focusing; thus, a separation in a pH value range between 5 and 7 could preferentially be achieved. A reactive spot with a molecular weight of 50 kD and an approximate pI value of 6.0 could be detected (Terjung, B. et al., Hepatology 28:332-340 (1998); Terjung, B. et al., Gastroenterology 119:310-322 (2000); U.S. Pat. No. 6,627,458). At this point, this spot was not analyzed in a mass spectrometric procedure. The interpretation of the results for this two-dimensional gel as well as for other gels was complicated by the inconstant pH gradients varying from gel to gel. At this point, nuclear envelope extracts from HL-60 cells were again separated with the currently used procedure of the two-dimensional gel electrophoresis (immobilized pH gradients, see also example no. 6) to compare the methods and for a better interpretation of the results. In addition to the reactive spots in the pH value range of 4.9 to 5.1 (tubulin beta isotype 5), even less reactive spots could be detected in the alkaline pH value range (5.5-6.4). Those are within the pI value range described in the US patent.

A mass spectrometric analysis of these reactive spots in the pH value range between 5.5 and 6.4 in an external sequencing laboratory resulted in signs of vimentin being the reactive protein (MW 55 kD, pI value 5.5; Mowse probability score: 328, a score of >45 is required). In summary, it can be ascertained that the isolation of an antigen fraction in accordance with the procedure presented in U.S. Pat. No. 6,627,458 did not show reliable results and could therefore not be used for the identification of the antigen as protein similar to tubulin beta isoform 5, since the protein mixture obtained by this procedure could not be further characterized by a sequencing procedure over MALDI-TOF or other procedures.

### Example 13

### Indirect Immunofluorescence Microscopy

The indirect immunofluorescence microscopy (IIF) as detection method for atypical p-ANCA was performed in accordance with standard protocols of the “First International Workshop on ANCA” (Wiik, A., APMIS 97(suppl.6): 12-13 (1989); Savige, J. et al., Am. J. Clin. Pathol. 111:507-513 (1999)). Commercial microscope slides with ethanol-fixed human neutrophils were used (INOVA Diagnostics). After purifying the microscope slide with PBS for several times, a secondary antibody conjugated with fluorescein was added (affinity-purified polyclonal conjugated fluorescein isothiocyanate (FITC) Goat Anti Human IgG), incubated for 30 minutes at room temperature in a humidity compartment, and finally purified with PBS for several times. After having applied an anti-bleaching agent, the microscope slides were examined through an epifluorescence microscope with 60× and 100× oil immersion lenses (Leica, Wetzlar, Germany). All staining patterns were independently evaluated by two examiners who did not know the clinical diagnose of the patients. Double stainings with a nuclear stain (e.g. propidium iodide) or antibodies against special subcellular components (e.g. nuclear envelope proteins, tubulin beta polypeptides) were used for the further characterization of the subcellular localization of the antigen.

### Example 14

### Cloning the FtsZ-Specific cDNA and Expression as Recombinant Protein

First, genomic DNA was extracted from E. coli (M15) for the production of recombinant FtsZ (MiniPrep, Qiagen). For this, 2 ml of an over night culture

of E. coli were centrifugated for 5 min. at 3,000 rpm. The obtained pellet was resuspended with 500 μl of a solution from 10 mM of Tris HCl (pH value 8.0) and 1 mM of EDTA (pH value 8.0); then, the bacteria were lysed by adding 500 μl of a solution consisting of 0.2 M of NaOH and 1% (w/v) SDS (60 min. at room temperature) and neutralized by adding a solution (700 μl) from 3 M of potassium acetate (pH value 4.8). After the centrifugation (10 min., 13,000 rpm), the solution was added to 700 μl of isopropanol and centrifugated again for 2-3 minutes at 13,000 rpm. Then, the dried pellet was added to 50 μl of H₂O/RNAse (100 μg/ml).

Subsequently, the obtained cDNA was amplified with the help of PCR (HotStarTaq DNA Polymerase, Qiagen, Denaturation: 94° C. 30 s Elongation: 51/62° C. 45 s, Synthesis: 72° C. 60 s); Primer [0.5 μM per preparation]:

| | |
|---|---|
| forward [NcoI Site]: | |
| 5′-cgccatggcgcgtatggcattcgcggagca-3′ | (SEQ ID NO:19) |
| backwards [XhoI Site]: | |
| 5′-cgctcgagatcagcttgcttacgcagg-S′. | (SEQ ID NO:18) |

The obtained genetic products were purified (QIAquick PCR Purification Kit, Qiagen). The expression vector pQE-TriSystem (Qiagen) was cut over specific restriction enzyme interfaces (NcoI and XhoI). Then, the used enzymes were inactivated for 10 min. at a temperature of 65° C., and the obtained vector fragments were purified (GeneElute Gel Purification Kit [Sigma]). The cDNA fragments of FtsZ were ligated into the pQE Tris vector (60 min., 22° C.) and then, E. coli was transformed with this vector (Rapid DNA Ligation & Transformation Kit, Fermentas). The suspension from E. coli and plasmid was incubated on an LB agar plate over night (37° C.). Grown clones were picked and cultivated at a temperature of 37° C. in LB medium which had been mixed with Amp/Kan (final concentration of ampicillin 100 ng/ml, final concentration of kanamycin 25 μg/ml). The cDNA of the produced colonies was then reisolated in accordance with the procedure described above, and the obtained plasmids were checked on a 2% agarose gel after the restriction digestion. The transformed E. coli were cultivated over night in 10 ml of LB Amp/Kan at a temperature of 37° C. After having added 90 ml of LB Amp/Kan, the transformed E. coli were further cultivated until reaching an optical density (OD) of 0.6. Subsequently, the expression of the cDNA of FtsZ was induced with 1 mM of isopropyl beta-D-thiogalactopyranoside (IPTG) (5 hours at a temperature of 37° C.). The obtained fusion protein marked with a polyhistidine tag was extracted with the help of nickel exchange resins, added to sample buffers and tested for its reactivity

with antibodies directed against the integrated polyhistidine tag, with antibodies directed against an FtsZ peptide or with sera containing atypical p-ANCA with the help of a one-dimensional or a two-dimensional gel electrophoresis or a subsequent immunodetection.

The used antibodies directed against an FtsZ peptide were polyclonal. The following peptide (14 AS) was used for their production:

(SEQ ID NO:36)

Cys-Leu-Thr-Val-Gly-Val-Val-Thr-Lys-Pro-Ser-Phe-
Glu-Gly

This peptide is not located in the section of the GTP binding site which is similar for FtsZ and TBB-5. It is located in another highly-conserved section of FtsZ. A selection criterion using a database analysis for this peptide was that it is conserved in most bacteria strands and that the antibody produced this way should be directed against the FtsZ of different bacteria. The polyclonal antibody was produced by Eurogentec (Seraing, Belgium) and is used with an optimum dilution ration of 1:500 to 1:1,000.

### Example 15

### Immune Reactivity of Patient Sera Containing Atypical p-ANCA and Patient Sera Not Containing Atypical p-ANCA with FtsZ

After the expression of recombinantly produced FTsZ in E. coli M15 (vector pQETris) the produced polyhistidine-labeled fusion protein was eluted in a pH dependent fashion via a nickel exchange resin, which separates polyhistidine-labeled proteins from the remaining cell proteins, after the production of lysates of the transformed E-coli. The eluted proteins were then separated in the following one-dimensional or two-dimensional SDS PAGE electrophoresis (see also example no. 5 and 6) and either stained with Coomassie Blue or transferred to a nitrocellulose membrane with the semi-dry blotting procedure. The proteins bound on the nitrocellulose membrane, especially FtsZ, were then tested for their reactivity with atypical p-ANCA by incubation with sera of AIH patients containing atypical p-ANCA and with sera of patients not containing atypical p-ANCA. An antibody to the polyhistidine tag was used as control antibody. The reaction of atypical p-ANCA or of the control antibody with FtsZ was proved by immunodetection with subsequent chemiluminescence reaction (see example 5 and 6). Here, the majority of the tested sera (8 of 10) containing atypical p-ANCA showed a reactivity with a 42 kD protein. No reactivity could be detected for ANCA-negative sera (n=9) of patients with non-autoimmune liver diseases (e.g. alcoholic cirrhosis), intestinal

diseases which cannot be assigned to the chronic inflammatory intestinal diseases, or for sera of healthy persons. A mass spectrometric analysis in the two-dimensional gel electrophoresis of a reactive spot (approx. 42 kD, pH value 5.0) reacting with atypical p-ANCA confirmed FtsZ being a reactive protein.

### Example 16

### Immunological Detection of Primary Sclerosing Cholangitis (PSC) and Autoimmune Hepatitis (AIH) by Atypical p-ANCA and its Diagnostic Relevance

A) Patients: Sera of 35 patients with PSC and sera of 175 patients with AIH were used.

All patients suffering from a liver disease were tested for AIH with test protocols based on the diagnostic evaluation suggested by the “International Autoimmune Hepatitis Study Group” (Alvarez, F. et al., J. Hepatology 31:929-938 (1999)). According to this point system, a value of more than 15 points prior to the treatment (more than 17 points during or after the treatment) “definitely” indicated an AIH disease, while a value of 10-15 points prior to the treatment with immunosuppressive drugs (12-17 points during or after the treatment) “possibly” indicated an AIH disease. Exclusion criteria for the diagnosis of AIH were viral hepatitis (A, B, C, D, E), metabolic hepatic disorders (hemochromatosis, Wilson's disease, alpha 1 antitrypsin deficiency), excessive alcohol consumption (>25 g/die) or a recent consumption of hepatotoxic noxious substances.

The diagnoses of the patients suffering from PSC were based on established, clinical, endoscopic, histological, radiological, and serological criteria (Angulo, P. et al., J. Hepatol. 32:182-187 (2000); Wiesner, R. H. et al., in: Krawitt, E. L et al., Hrsg., Autoimmune Liver Diseases, 2nd Ed., Amsterdam, Elsevier, 381-412 (1998)). Other exclusion criteria were severe bacterial or viral infections within 7 days before the serum withdrawal, a neoplasm anamnesis, or a concomitant HIV or TBC infection.

The clinical and biochemical characteristics of the participants in the study at the time of the withdrawal of the serum sample are summarized in table 2. All data will be specified either as mean value±standard error of the mean value or as median. Until being used, the sera were stored at a temperature of −20° C. All steps of the study have been performed in accordance with the valid version of the Helsinki declaration

of 1975. The study has been approved by the Ethics Committee of the University of Bonn (Germany) responsible for the examination.

TABLE 2
Characteristics of the study participants

| | | | | | Alkohol- | Normal |
|---|---|---|---|---|---|---|
| | AIH | PSC | PBC | HCV | toxic LZ | controls |
| | (n = 175) | (n = 35) | (n = 45) | (n = 21) | (n = 62) | (n = 19) |
| Sex (f/m) | 119/56 | 6/29 | 37/8 | 8/13 | 36/26 | 12/7 |
| Median age in | 53 | 53 | 57 | 47 | 56 | 35 |
| years (range) | (10-78) | (21-67) | (26-79) | (27-73) | (36-78) | (24-61) |
| Biochemical parameters | | | | | | |
| (Mean values SEM) | | | | | | |
| aP (normal 40-190 U/ml) | 252 ± 129 | 432 ± 210 | 360 ± 214 | 143 ± 64 | 202 ± 13 | 88 ± 21 |
| ALT (normal 5-19 U/ml) | 206 ± 219 | 42 ± 20 | 165 ± 136 | 83 ± 47 | 25 ± 22 | 12 ± 6 |
| Bilirubin normal | 1.3 ± 1.1 | 4.9 ± 5.2 | 2.2 ± 2.1 | 0.8 ± 0.6 | 3.0 ± 3.3 | 0.4 ± 0.2 |
| <1, 1 mg/dl) | | | | | | |

Abbreviations: SEM: Standard error of mean; aP: alkaline phosphatase; ALT: Alanin transferase (GPT); AIM: autoimmune hepatitis; PSC: Primary sclerosing cholangitis; PBC: Primary biliary cirrhosis; HCV: Chronic infection with hepatitis C; Alcohol-toxic LC: alcohol-toxic liver cirrhosis

B) Detection of atypical p-ANCA, anti-nuclear antibodies (ANA), smooth muscle antibodies (SMA), anti-mitochondrial antibodies (AMA), and anti-liver and kidney microsomes (anti-LKM1) by indirect immunofluorescence microscopy: All detected antibodies, i.e. atypical p-ANCA, ANA, SMA, AMA and anti-LMK1 were proved with the help of the indirect immunofluorescence microscopy (see also example no. 13). For this, microscope slides with diluted sera were preincubated for 30 minutes at room temperature in a humidity compartment. After several purifying passes with PBS, bound antibodies were coupled to FITC-coupled, secondary antibodies according to the manufacturer regulations. Normally, this was accomplished by incubating the antibodies for 20 minutes at room temperature in the humidity compartment (secondary antibodies for the detection of ANA obtained from Innogenetics, Gent, Belgium; all other secondary antibodies obtained from INOVA Diagnostics, La Jolla, Calif.). The evaluation was accomplished as described in example no. 13.

Atypical p-ANCA. Atypical p-ANCA in sera were detected on ethanol-fixed neutrophil granulocytes (INOVA Diagnostics). There are three different staining patterns: c-ANCA with a diffuse cytoplasmic fluorescence, “classic” p-ANCA with a homogeneous, annular staining of the perinuclear cytoplasm, and “atypical” p-ANCA with an inhomogeneous annular staining of the nuclear periphery in connection with a characteristic intranuclear mottled pattern (Savige, J. et al., Am. J. Clin. Pathol. 111:507-513 (1999); Terjung, B. et al., Clin. Exp. Immunol. 126:37-46 (2001)). To exclude false positive results due to the simultaneous presence of ANA and ANCA, the serum titer for ANCA had to be four times higher than the serum titer for ANA.

ANA. Hep2 cells were used for the detection of ANA (Innogenetics) which resulted in a homogeneous, mottled, centromere, annular or nucleolus staining of the nucleus (Tan, E. M., Adv. Immunol. 44:93-151 (1989); Czaja, A. J. et al., Gastroenterology 107:200-207 (1994)). ANA serum titers higher or equal to 1:40 were positively valued (Alvarez, F. et al., J. Hepatol. 31:929-938 (1999)).

SMA, AMA and anti-LKM1. Cryostat sections of shock frozen liver, kidney, and stomach of rats or mice were used for the detection of SMA, AMA and/or anti-LKM1 (INOVA Diagnostics) (Toh, B. H., Clin. Exp. Immunol. 38:621-628 (1979); Czaja, A., et al., Gastroenterology 107:200-207 (1994); Manns, M. P. et al., J. Clin. Invest. 83:1066-1072 (1989)). The fluorescence pattern of SMA was characterized by the staining of muscle fibers of the blood vessels, the gastric mucosa and the lamina propria fibers of the stomach. AMA showed a staining of the mitochondria in distal tubules of the kidney and in gastric parietal cells. In contrast, anti-LKM1 did not stain gastric parietal cells and strongly reacted with microsomal proteins of the proximal kidney tubules and with cytoplasmic structures of the murine liver cells. Serum titers of SMA, AMA and anti-LKM1 higher or equal to 1:80 were positively valued.

C) Detection of antibodies to soluble liver antigen (anti-SLA/LP), anti-LKM1, and antibodies to M2 (anti-M2) by ELISA: All ELISAS were used according to the manufacturers' specifications. Patient sera and calibration sera of the manufacturers were diluted with PBS at a ratio of 1:100. The absorption values were determined with a photometer at 450 nm (Spectra Mini, Tecan, Groedig, Austria).

Anti-SLA/LP. Two ELISAS of different manufacturers (Euroimmun, Lubeck, Germany; INOVA Diagnostics) were used for the detection of anti-SLA/LP. Both ELISAS used human SLA/LP antigen. A ratio higher than 1.0 or 20 units/ml (Euroimmun) or 25 units/ml (INOVA Diagnostics) respectively, was positively valued.

Anti-LKM1. Human recombinant cytochrome P450 IID6 was used for the detection of anti-LKM1 (Varelisa anti-LKM1, Pharmacia Diagnostics, Freiburg, Germany). Thresholds higher or equal to 5 units/ml were positively valued.

Anti-M2. Affinity-purified pyruvate dehydrogenase complexes from mitochondria were used as antigen (Varelisa anti M2, Pharmacia Diagnostics). Values higher or equal to 5 U/ml indicated the presence of anti-M2.

D) Statistics: Data were either specified as median or as mean value±standard error (SEM, standard error of mean). Fischer's test of significance was used for the comparison of categorical data. Continuous data were examined with the non-parametric unpaired Wilcoxon test. The normal distribution of the parameters was determined with the Kolmogorov-Smirnov test. If required, a logarithmic transformation was performed to obtain a normal distribution. To characterize each test regarding its diagnostic accuracy, sensitivity and specificity, and to characterize its optimal predilutions and thresholds, statistical parameter were calculated with the help of 2×2 contingency tables for each possible threshold and applied as “receiver operator curves” (ROC). The surface below the ROC curve (AUC) and its SEM were calculated with the Medcalc software (version 7.1, [MedCalc, Mariakerke, Belgium]) and, if required, with the SPSS software (version 11.0, [SPSS Inc., Chicago, Ill.]). The significance of the AUC was analyzed with the “standard critical ratio test”. z=(W-0.5)/SE_{w} (z=critical ratio; W=probability; SE_{w}=standard error of W). This evaluation checks the hypothesis saying that the AUC does not significantly differ from 0.5 which corresponds to the surface below the 450 diagonal line indicating the indistinction between normal and abnormal. The critical ratio z was calculated for the statistical comparison of two ROC curves A and B in the following way: Z=(AUC_{A}−AUC_{B})/[(SE_{A})₂−2 r SE_{A} SE_{B}], where r is the estimated correlation between AUC_{A} and AUC_{B} (Hanley, J. A: und McNeil, B. J., Radiology, 148:839-843 (1983); Zweig, M. H. und Campbell, G., Clin. Chem. 39:561-577 (1993)). The results of both z statistics were specified as bilateral p values. Optimal thresholds (parameter determined by ELISA, serum titer determined

by IIF) corresponded with the highest-precision values (slightly false negative and false positive results). Variables (atypical p-ANCA, ANA, SMA, AMA, anti-SLA/LP, anti-M2, anti-LKM1 [determined by IIF or ELISA] were stepwise entered into a multiple, conditional, logistic forward regression analysis using SPSS software (version 11.0) to determine independent factors which are connected with the diagnosis of PSC and AIH. The inclusion or exclusion criteria for entering variables into the final model were p<0.05 or p>0.10. The value of the regression coefficient Exp(B) was used for all significant variables in the multiple analysis to calculate the respective odds ratios (e_{ExP}(B)) and their 95% confidence intervals (Bland, J. M. und Altman, D. G., BMJ 320:1468 (2000)). Provided that only antibodies and their serum titers determined through IIF were entered into the regression model, the serum titers were sequentially encoded: 1:10 corresponded to 1, 1:20 to 2, 1:40 to 3, 1:80 to 4 etc. Thus, each odds ratio reflects the influence of an independent variable (e.g. an antibody) on the diagnosis of a given disease related to a certain serum titer of the antibody. However, if ELISA results in units/ml were considered together with serum titers according to IIF, only the presence or absence of a parameter was encoded and entered into the regression model. Thus, the calculated odds ratios reflected the influence of the presence or absence of an independent variable on the diagnosis of a certain disease.

E) Results: The results have been summarized in tables 3 and 4.

TABLE 3
Antibody prevalence for AIH, PSC, PBC and
AIH/PSC or AIH/PBC overlap syndromes.

| | AIH | PSC | PBC | AIH/PSC | AIH/PBC |
|---|---|---|---|---|---|
| | (n = 175) | (n = 35) | (n = 45) | (n = 9) | (n = 11) |
| Atypical p-ANCA | 81% | 94% | 31% | 100% | 73% |
| (IIF) | | | | | |
| ANA (IIF) | 61% | 43% | 53% | 78% | 64% |
| SMA (IIF) | 48% | 40% | 9% | 44% | 18% |
| Anti-Lkm1 (IIF) | 5% | 0% | 0% | 0% | 0% |
| Anti-Lkm1 | 4% | 0% | 0% | 0% | 0% |
| (ELISA) | | | | | |
| AMA (IIF) | 4% | 0% | 62% | 0% | 27% |
| Anti-M2 (ELISA) | 14% | 0% | 69% | 0% | 36% |
| Anti-SLA/LP | 17% | 0% | 0% | 11% | 0% |
| (ELISA) | | | | | |

Abbreviations: AIH/PSC: Overlap syndrome between autoimmune hepatitis and PSC; AIH/PBC: Overlap syndrome between AIH and PBC; ANA: Antinuclear antibodies; SMA: Smooth muscle antibodies; Anti-LKM1: Anti-liver and kidney microsomes; AMA: Antibodies to mitochondrial antigen (E2 subunit of the pyruvate dehydrogenase complex); Anti-M2: Antibodies to the E2 subunit of the pyruvate dehydrogenase complex; Anti-SLA/LP: Antibodies to soluble liver antigen/liver pancreas antigen; IIF: Indirect immunofluorescence microscopy; ELISA: Enzyme linked immunosorbent assay.

TABLE 4
Determination of diagnostically relevant autoantibodies
with regard to the underlying diseases with the help of
a forward conditional logistic regression analysis.

| | | | | |
|---|---|---|---|---|
| | | | | True |
| P | | Odds | 95% CI of the | posi- |
| value¹ | Exp(B)² | ratio² | odds ratio² | tive³ |

AIH

| | | | | | |
|---|---|---|---|---|---|
| Atyp- | 0.001 | 1.21 | 3.36 | 3.00-3.82 | 73% |
| ical p- | | | | | |
| ANCA⁴ | | | | | |
| SMA⁴ | 0.001 | 1.72 | 4.05 | 3.22-5.30 | 72% |
| ANA⁴ | 0.839 | — | — | — | — |
| Anti- | 0.001 | 17.18 | 28 × 10⁶ | 0-∞ | 74% |
| SLA/ | | | | | |
| LP⁵ | | | | | |
| Anti- | 0.003 | 4.55 | 95.01 | 0-∞ | 75% |
| Lkm1 | | | | | |
| (IIF)⁴ | | | | | |

PSC

| | | | | | |
|---|---|---|---|---|---|
| Atyp- | 0.000 | 1.22 | 3.39 | 3.03-3.85 | 90% |
| ical p- | | | | | |
| ANCA⁴ | | | | | |

PBC

| | | | | | |
|---|---|---|---|---|---|
| AMA⁴ | 0.000 | 9.84 | 18.8 × 10³ | 206.64-7.7 × 10⁷ | 93% |
| Anti- | 0.001 | 5.36 | 212.72 | 5.75-13.96 × 10⁶ | 92% |
| M2⁵ | | | | | |

¹A p value <0.05 was considered as being significant.
²Exp(B) was used to calculate the respective odds ratios (e^{Exp(B)}) and their 95% confidence interval (CI). Odds ratios higher than 1.0 were positively valued.
³True positives in %: Patient with a correct diagnosis if the respective antibody was detected.
⁴Serum titers determined by IIF were sequentially encoded:
⁵The results of the ELISA were either positively (=1) or negatively (=0) encoded. Thus, the odds ratios reflect the influence of an existent or non-existent independent variable on the diagnosis of a certain disease.

1. Method for identifying and/or quantifying atypical anti-neutrophil cytoplasmic antibodies (atypical p-ANCA) in a sample comprising
providing an isolated antigen for bonding with atypical p-ANCA, and
identifying and/or quantifying atypical P-ANCA in said sample.

2. Method according to claim 1, wherein the antigen is a protein having a molecular weight of about 50 kD, determined by SDS PAGE gel electrophoresis, and an isoelectric point (IEP) of pH value 4.7-5.1, determined by isoelectric focusing.

3. Method according to claim 1, wherein the antigen is a protein including
(i) partial sequence ALTVPELTQQVFDAK (SEQ ID NO:3), and/or
(ii) one or more of partial sequences, selected from SEQ ID NO:20, SEQ ID NO:21, SEQ ID NO:22, SEQ ID NO:23, SEQ ID NO:24, SEQ ID NO:25, SEQ ID NO:26 and SEQ ID NO:27.

4. Method according to claim 1, wherein
(i) the antigen is isolated from tubulin preparations from human cells or vertebrate cells, and/or
(ii) the antigen is isolated from nuclear envelope preparations of myeloid differentiated cells.

5. Method according to claim 1, wherein the antigen is
(i) a tubulin beta, and/or
(ii) a TBB-5 derivate, wherein a native amino acid sequence of TBB-5 is truncated at a N terminus and/or a C terminus by 1-220 amino acids or by 1-100 amino acids or by 1-50 amino acids, and/or
(iii) a protein with SEQ ID NO:5, SEQ ID NO:7 or SEQ ID NO:9.

6. Method according to claim 1, wherein the antigen is a bacterial cell division protein.

7. Method according to claim 1, wherein the antigen forms a fusion protein with a non-toxic, directly or indirectly detectable marker protein, and wherein the marker protein is
(i) a functional, calorimetrically detectable protein; and/or
(ii) a peptide which facilitates isolation of the fusion protein.

8. Method according to claim 7, wherein the antigen is a GFP fusion protein of TBB-5 and preferably has the amino acid sequence of SEQ ID NO:29.

9. Method according to claim 1, wherein
(i) the atypical p-ANCA shows an epitope for a secondary agent, and/or
(ii) the sample comprises an appropriate human body fluid.

10. Method according to claim 9, wherein the secondary agent
(i) is specific for an epitope on atypical p-ANCA, and/or
(ii) is a human, murine, rat, rabbit, goat, sheep, or pig antibody, and/or
(iii) is coupled with a detector reagent allowing detection of the antibody as well as the quantitative and qualitative evaluation of a bond of the secondary agent.

11. Method according to claim 1, wherein
(i) the sample is brought into contact with the antigen to atypical p-ANCA so that atypical p-ANCA existent in the sample are bound to the antigen to atypical p-ANCA, and/or
(ii) the bound atypical p-ANCA or the bound p-ANCA antigen is detected by immunological methods, and/or
(iii) the p-ANCA titer of the sample is determined by immunological methods, and/or
(iv) the antigen to atypical p-ANCA is bound to a carrier material by an adequate coupling technique which does not modify specificity of the antigen for atypical p-ANCA, and/or
(v) the procedure is performed in vitro.

12. Method according to claim 1, further comprising quantitative
determination of a concentration of atypical p-ANCA in a sample
(i) via an immunoassay, and/or
(ii) via a comparison with a standard concentration of atypical p-ANCA.

13. Method according to claim 1, wherein chronic inflammatory intestinal diseases, including ulcerative colitis and/or Crohn's disease, and/or autoimmune liver diseases, including autoimmune hepatitis and/or primary sclerosing cholangitis are detectable.

14. Protein for binding atypical p-ANCA and as defined in claim 2, but excluding TBB-5 with an amino acid sequence of SEQ ID NO:2 or FtsZ with an amino acid sequence of SEQ ID NO:17.

15. DNA encoding for a protein according to claim 14.

16. A vector including a DNA according to claim 15.

17. A host organism which is transformed/transfected with a vector according to claim 16.

18. Method for the production of a protein according to claim 14 comprising cultivation of a host organism, wherein the host organism is transformed/transfected with a vector including a DNA encoding said protein.

19. Kit for the identification and/or quantification of atypical p-ANCA according to the method of claim 1 including
(i) an isolated antigen for bonding with atypical p-ANCA, and identifying and/or quantifying atypical P-ANCA in a sample, and/or
(ii) a microbial strain of a cell line, which is able to express said antigen.

20. Kit according to claim 19, wherein
(i) the antigen to atypical p-ANCA is bound to a carrier material, and/or
(ii) the kit further includes
a secondary agent as defined in claim 10,
agents for the detection of a bond of atypical p-ANCA to the secondary agent via the antigen,
buffers and/or culture media.

21. Immunological, neurobiological and cell physiological analyses comprising providing the protein of claim 14, in clinical research as well as for diagnostic methods in vivo and in vitro.

22. Method according to claim 3, wherein said one or more partial sequences in (ii) are selected from SEQ ID NO:20, SEQ ID NO:21, SEQ ID NO:22 and SEQ ID NO:23.

23. Method according to claim 4, wherein the antigen in (i) is isolated from tubulin preparations from blood cells, cells of the immune system, from hepatocytes, cells of the bile system, or intestinal cells.

24. Method according to claim 4, wherein the antigen in (i) is isolated from tubulin preparations from myeloid differentiated cells or neutrophil granulocytes.

25. Method according to claim 5, wherein the tubulin beta in (i) is a TBB-5 with amino acid sequence SEQ ID NO:2 or a substitution mutant, a deletion mutant and/or an addition mutant thereof.

26. Method according to claim 6, wherein the bacterial cell division protein is bacterial cell division protein FtsZ with amino acid sequence SEQ ID NO:17, or a substitution mutant, a deletion mutant and/or an addition mutant thereof.

27. Method according to claim 26, wherein the bacterial cell division protein FtsZ with amino acid sequence with SEQ ID NO:36, amino acid sequences of positions 65-135 of SEQ ID NO: 17 and/or the amino acid sequence of positions 255-300 of SEQ ID NO:17.

28. Method according to claim 7, wherein the marker protein in (i) is a fluorescent protein, and/or the peptide in (ii) is a His tag, a c-myc tag or an Xpress tag.

29. Method according to claim 28, wherein the fluorescent protein is a GFP or a mutant thereof.

30. Method according to claim 9, wherein the secondary agent in (i) is an antibody and/or the human body fluid in (ii) is blood or serum.

31. Method according to claim 10, wherein the detector agent is selected from enzymes, fluorescence stains and radio isotopes, especially preferably horseradish peroxidase or fluorescein.

32. Method according to claim 10, wherein the secondary agent is a polyclonal fluorescein isothiocyanate (FITC)-conjugated Goat Anti Human IgG or a horseradish peroxidase-coupled Sheep Anti Human IgG.

33. Method according to claim 11, wherein said coupling technique in (iv) is a covalent cross-linking technique.

34. Method according to claim 12, wherein said immunoassay is an ELISA.
